(19) United States
(12) Patent Application Publication
(10) Pub. No.: US 2024/0103011 A1
(43) Pub. Date: Mar. 28, 2024

(54) LIQUID BIOPSY YIELD ENHANCEMENT

(71) Applicants: Seema Singhal, Chicago, IL (US); Jayesh Mehta, Chicago, IL (US); Neil Mehta, Philadelphia, PA (US); Aran Mehta, Chicago, IL (US)

(72) Inventors: Seema Singhal, Chicago, IL (US); Jayesh Mehta, Chicago, IL (US); Neil Mehta, Philadelphia, PA (US); Aran Mehta, Chicago, IL (US)

(21) Appl. No.: 18/273,384

(22) PCT Filed: Jan. 20, 2022
(86) PCT No.: PCT/US2022/013189

§ 371 Date: Jul. 20, 2023

Related U.S. Application Data
(60) Provisional application No. 63/236,926, filed on Aug. 25, 2021.
(60) Provisional application No. 63/189,614, filed on May 17, 2021.
(60) Provisional application No. 63/139,561, filed on Jan. 20, 2021.

(51) Int. Cl. G01N 33/68

(52) U.S. Cl. CPC G01N 33/6863; G01N 2800/52; G01N 2800/54

(57) ABSTRACT

Provided herein are methods, kits, systems, and compositions or liquid biopsy yield enhancement.

### CROSS REFERENCE TO RELATED APPLICATIONS

This application claims the benefit of, and priority to, U.S. Provisional Application No. 63/139,561, filed on Jan. 20, 2021; U.S. Provisional Application No. 63/189,614, filed on May 17, 2021; and U.S. Provisional Application No. 63/236,926, filed on Aug. 25, 2021, the contents of each of which are hereby incorporated by reference in their entirety for all purposes.

### BACKGROUND

Disease detection, diagnostic, and prognostic methods often rely on sampling of diseased cells, e.g., cancer cells. In many cases, tissue biopsies to obtain such cells can be invasive and painful, and carry risks of surgical complications. For example, in particular cases, such as multiple myeloma, cancerous cells are obtained via a puncture of the bone (bone marrow biopsy), a procedure that is extremely unpleasant for patients. In many cases, access to diseased tissue is difficult, further complicating sampling of diseased cells for accurate diagnosis.

In some cases, distribution of diseased cells within the patient is patchy, further complicating biopsy and diagnosis.

A considerable number of patients with seemingly successful cancer treatment, e.g., complete tumor resection or exhibiting complete remission (CR) after treatment suffer relapse within a variable period of time. Such relapses demonstrate the presence of undetected micrometastases or minimal/molecular residual disease (MRD) even after seemingly successful therapy. Presence of MRD is predictive of relapse (relapse signifying presence of disease that is detectable by conventional techniques) and is an important prognostic factor in several malignancies. Indeed, a major practical application of MRD detection is the administration of cancer therapy in a subject if MRD is detected to avert full-blown relapse (i.e., relapse detectable clinically and/or by conventional tests) and improve the probability of cure or long-term survival.

The difficulties in detecting MRD are complicated by the factors mentioned above, such as patchy distribution of and difficulty in accessing malignant cells.

Cancer treatment often suffers from a lack of long-term efficacy. Tumors can become resistant to therapy, at least in part by the accumulation of genetic aberrations that may not have been present during initial characterization and therapy. For example, in the case of multiple myeloma, there is often spatiotemporal heterogeneity in mutational status of focal lesions.

However, current practice for diagnosis and prognosis involves sampling from a single site sequentially or different sites at different times, thereby missing an opportunity for complete profiling of the patient's cancer. Standard liquid biopsy and sequencing analysis of cell-free DNA from multiple myeloma patients, by showing heterogeneity in mutational profiles compared with concomitantly analyzed bone marrow samples, has demonstrated the utility of standard liquid biopsy in tumor profiling of cancers having high spatiotemporal heterogeneity in mutational profiles.

Liquid biopsy techniques have been advanced over the past several years for the detection of circulating tumor cells (CTCs) and/or circulating tumor DNA (ctDNA) from liquid samples obtained from patients, e.g., blood samples. Liquid biopsies confer many advantages over traditional biopsy, being generally painless, non-invasive, and convenient. Detection of CTC or ctDNA after seemingly complete surgical tumor resection has been correlated with poor clinical outcomes, often preceding overt cancer recurrence by months.

However, liquid biopsy techniques suffer from inadequate sensitivity, resulting in false negatives. For example, a ctDNA study using plasma samples from cancer patients exhibited sensitivity that varied from 59% in early lung cancer up to 86% in early head and neck cancers. The ctDNA study demonstrated sensitivity rates of 34% for stage I cancer, 77% for stage II, 84% for stage III, and 92% for stage IV across all tumor types (Liu et al 2019). Such results indicate that current liquid biopsy applications do not have the very high degree of sensitivity required for MRD detection in a significant proportion of patients. Such inadequate sensitivity likely stems from the low number of CTCs or ctDNA released into circulation during MRD states. Attempts to amplify manifold possible tumor material already present in the fluid being analyzed to make the cancer detectable runs the risk of false positive findings.

In addition to disease diagnosis and prognosis, liquid biopsy may also be used to uncover information that may help choose therapy for a patient's cancer in a targeted and more precise fashion. The pieces of information in a liquid biopsy result that may be utilized to make therapeutic decisions include, but are not limited to, the presence of mutations/genomic variants (whether “actionable” or not), the proportion of cells/DNA material exhibiting abnormality or change (variant allele fraction), tumor mutational burden (TMB), microsatellite instability (MSI), fragmentomes, telomere length, thymidylate synthase expression, and DNA methylation patterns.

There exists a need for safe methods and systems for enhancing sensitivity of liquid biopsy techniques, in order to reduce false negatives in disease detection, reducing the risk of false positives by excessive amplification of signals present in liquid biopsy specimens, and to improve prognosis. Such methods and systems would be particularly useful for, e.g., enhancing diagnostic yields in individuals with no detectable cancer, MRD detection, disease prognosis, and early disease detection. For example, such methods and systems would be useful to make mass screening of otherwise normal individuals a realistic possibility with a high level of sensitivity that could enable identification of cancer when it's very early—and therefore, by definition, much more treatable/curable. There also remains a need for safe methods and systems to obtain or to increase the possibility to obtain clinically or therapeutically useful (“actionable”) information from a liquid biopsy to guide personalized cancer treatment.

### SUMMARY OF THE INVENTION

In various embodiments, the present disclosure provides a method of analyzing a fluid sample obtained from a subject, comprising determining the presence or absence of one or more diseased cells, disease-associated exosomes, disease-associated transcriptomes, or disease-associated polynucleotides in the fluid sample, wherein the subject has been administered an effective amount of one or more cytokines, chemokines, growth factors, or coagulation system modulators, or combinations thereof. In various embodiments, administering one or more cytokines, chemokines, or growth factors, or combinations thereof, as described herein, modulates surface markers, proteins, organelles, or metabolic processes involving cancer cells, their environment (including normal cells and extracellular fluid such as plasma), or the interactions thereof, and/or liberates cells or material into the circulation.

In another aspect, provided herein is a method of analyzing a fluid sample obtained from a subject, comprising determining the presence or absence of one or more diseased cells, disease-associated exosomes, disease-associated transcriptomes, or disease-associated polynucleotides in the fluid sample, wherein the subject has been administered an effective amount of one or more cytokines, chemokines, or growth factors or coagulation system modulators. In one aspect, provided herein is a method of detecting one or more diseased cells, disease-associated exosomes, disease-associated transcriptomes, or disease-associated polynucleotides in a fluid sample obtained from a subject, comprising: a. administering to the subject an effective amount one or more cytokines, chemokines, or growth factors or coagulation system modulators; b. obtaining the fluid sample from the subject; and c. determining the presence or absence of one or more diseased cells, disease-associated exosomes, disease-associated transcriptomes, or disease-associated polynucleotides in the fluid sample, wherein, if the detection is the detection of diseased cells or disease-associated polynucleotides, the cytokine is not plerixafor.

In another aspect, provided herein is a method of identifying a disease in a subject in need thereof, comprising detecting one or more diseased cells, disease-associated exosomes, disease-associated transcriptomes, or disease-associated polynucleotides in a fluid sample obtained from the subject, wherein the subject has been administered an effective amount of one or more cytokines, chemokines, or growth factors or coagulation system modulators, and wherein the detection of the one or more diseased cells, disease-associated exosomes, disease-associated transcriptomes, or disease-associated polynucleotides is indicative of the disease in the subject.

In another aspect, provided herein is a method of detecting a disease in a subject in need thereof, comprising: a. administering to the subject an effective amount of one or more cytokines, chemokines, or growth factors or coagulation system modulators; b. obtaining a fluid sample from the subject; and c. detecting the presence or absence of one or more diseased cells, disease-associated exosomes, disease-associated transcriptomes, or disease-associated polynucleotides in the fluid sample, wherein the detection of the presence of one or more diseased cells, disease-associated exosomes, disease-associated transcriptomes, or disease-associated polynucleotides is indicative of the disease in the subject, and wherein, if the detection is the detection of diseased cells or disease-associated polynucleotides, the cytokine is not plerixafor.

In another aspect, provided herein is a method of prognosing a subject in need thereof, comprising omics profiling one or more diseased cells, disease-associated exosomes, disease-associated transcriptomes, or disease-associated polynucleotides in a fluid sample obtained from the subject, wherein the subject has been administered an effective amount of one or more cytokines, chemokines, or growth factors or coagulation system modulators, and wherein the genetic profile of the one or more diseased cells, disease-associated exosomes, disease-associated transcriptomes, or disease-associated polynucleotides is indicative of the subject's prognosis.

In another aspect, provided herein is a method of prognosing a subject in need thereof, comprising: a. administering to the subject an effective amount of one or more cytokines, chemokines, or growth factors or coagulation system modulators; b. obtaining a fluid sample from the subject; and c. detecting one or more diseased cells, disease-associated exosomes, disease-associated transcriptomes, or disease-associated polynucleotides in the fluid sample, wherein the detection of diseased cells, disease-associated exosomes, disease-associated transcriptomes, or disease-associated polynucleotides is indicative of the subject's prognosis, wherein, if the detection is the detection of diseased cells or disease-associated polynucleotides, the cytokine is not plerixafor.

In some embodiments, the one or more cytokines, chemokines, or growth factors comprises a CXCR4 antagonist, a growth-related gene product β (GROβ) or a fragment or analog thereof, or a combination thereof. In some embodiments, the one or more coagulation system modulators comprises heparin or a derivative thereof, a direct oral anticoagulant (DOAC), a tissue plasminogen activator (tPA), a streptokinase, an urokinase, or a plasminogen activator inhibitor-1 (PAI-1) modulator.

In some embodiments, the steps of the provided methods are performed in the order of step a to step c.

In another aspect, provided herein is a method of predicting disease recurrence in a subject, comprising analyzing and determining change of quantity in diseased cells, disease-associated exosomes, disease-associated transcriptomes, or disease-associated polynucleotides in a first sample and a second sample obtained from the subject, wherein the subject is administered an effective amount of an intervening agent, after the first sample is obtained and before the second sample is obtained.

In another aspect, provided herein is a method of predicting disease recurrence in a subject, comprising the steps of: a. obtaining a first fluid sample from the subject and analyzing the quantity of diseased cells, disease-associated exosomes, disease-associated transcriptomes, or disease-associated polynucleotides; b. administering to the subject an effective amount an intervening agent; c. obtaining a second fluid sample from the subject and analyzing the quantity of diseased cells, disease-associated exosomes, disease-associated transcriptomes, or disease-associated polynucleotide; and; d. determining change of quantity in the diseased cells, disease-associated exosomes, disease-associated transcriptomes, or disease-associated polynucleotides in the first and the second fluid samples; thereby predicting disease recurrence by evaluating the change.

In some embodiments, the subject is administered an effective amount of an intervening agent before the first sample is obtained.

In some embodiments, the fluid sample is a blood sample. In some embodiments, the blood sample is a plasma or serum sample. In some embodiments, the blood sample is a whole blood sample or a cellular fraction of a whole blood sample. In some embodiments, the fluid sample is an ascites, cerebrospinal fluid, lymph, sweat, urine, tears, saliva, pleural fluid, pericardial fluid, bronchoalveolar fluid, cavity rinse or swab, or organ rinse or swab sample.

In some embodiments, the presence or absence or quantity of one or more diseased cells, disease-associated exosomes, disease-associated transcriptomes, or disease-associated polynucleotides in the fluid sample is determined using one or more methods selected from: flow cytometry, PCR (e.g., qPCR or ASO-qPCR), sequencing (e.g., next-generation sequencing, single-cell sequencing), immunostaining, immunohistochemistry, or immunofluorescence. In some embodiments, the presence or absence or quantity of one or more diseased cells, disease-associated exosomes, disease-associated transcriptomes, or disease-associated polynucleotides in the fluid sample is determined using a method with a sensitivity of at least 1 in 100,000 cells.

In another aspect, provided herein is a method of obtaining actionable information in a subject, comprising analyzing and determining change of a biomarker in a first sample and a second sample obtained from the subject, wherein the subject is administered an effective amount of an intervening agent, after the first sample is obtained and before the second sample is obtained.

In another aspect, provided herein is a method of obtaining actionable information in a subject, comprising the steps of: a. obtaining a first sample from the subject and analyzing a biomarker; b. administering to the subject an effective amount of an intervening agent; c. obtaining a second sample from the subject and analyzing the biomarker; and d. determining change of the biomarker in the first and the second samples, thereby obtaining actionable information.

In some embodiments, the analysis of the biomarker obtained from the first sample does not yield actionable information. In some embodiments, the actionable information is different from information obtained from analyzing the biomarker obtained from the first sample.

In another aspect, provided herein is a method of predicting treatment response of a subject, comprising analyzing and determining change of a biomarker in a first sample and a second sample obtained from the subject, wherein the subject is administered an effective amount of an intervening agent, after the first sample is obtained and before the second sample is obtained.

In another aspect, provided herein is a method of predicting treatment response of a subject, comprising the steps of: a. obtaining a first sample from the subject and analyzing a biomarker; b. administering to the subject an effective amount of an intervening agent; c. obtaining a second sample from the subject and analyzing the biomarker; and d. determining change of the biomarker in the first and the second samples, thereby predicting treatment response by evaluating the change of the biomarker. In some embodiments, the prediction of treatment response is different from a prediction made from analyzing the biomarker obtained from the first sample.

In another aspect, provided herein is a method of supporting treatment decision of a subject, comprising analyzing and determining change of a biomarker in a first sample and a second sample obtained from the subject, wherein the subject is administered an effective amount of an intervening agent, after the first sample is obtained and before the second sample is obtained.

In another aspect, provided herein is a method of supporting treatment decision of a subject, comprising the steps of: a. obtaining a first sample from the subject and analyzing a biomarker; b. administering to the subject an effective amount an intervening agent; c. obtaining a second sample from the subject and analyzing the biomarker; and d. determining change of the biomarker in the first and the second samples; thereby making an optimal treatment decision by evaluating the change of the biomarker.

In some embodiments, the optimal treatment decision is different from a decision made from analyzing the biomarker obtained from the first sample. In some embodiments, the treatment decision is starting a treatment, staying on current treatment, adjusting current treatment, stopping treatment, or switching to a different treatment.

In some embodiments, steps of a method provided herein are performed in the order of step a to step d.

In some embodiments, the biomarker is not detectable from the first sample. In some embodiments, biomarker is a hormone, a protein, a gene, a gene mutation, a genetic amplification or translocation, a mutational or genetic profile, a genome-wide fragmentation profile, variant allele frequency (VAF), tumor mutational burden (TMB), microsatellite instability (MSI), DNA repair deficiency/defect, DNA methylation pattern, or dysbiosis. In some embodiments, analyzing the biomarker is determining presence or absence of the biomarker or determining qualitative and quantitative data of the biomarker. In some embodiments, analyzing the biomarker is determining presence or absence of a protein, a gene, a gene mutation, or dysbiosis, determining telomere length, thymidylate synthase expression, or hypomethylation, determining quantitative data of VAF or TMB, or determining qualitative data of MSI. In some embodiments, the biomarker is analyzed using one or more methods selected from: flow cytometry, PCR (e.g., qPCR or ASO-qPCR), sequencing (e.g., next-generation sequencing, single-cell sequencing), immunostaining, immunohistochemistry, or immunofluorescence.

In some embodiments, the first sample obtained from the subject may is a tissue sample or a fluid sample. In some embodiments, the second sample is obtained from the subject is a fluid sample. In some embodiments, the fluid sample is a blood sample. In some embodiments, the blood sample is a plasma or serum sample. In some embodiments, the blood sample is a whole blood sample or a cellular fraction of a whole blood sample. In some embodiments, the fluid sample is an ascites, cerebrospinal fluid, lymph, sweat, urine, tears, saliva, pleural fluid, pericardial fluid, bronchoalveolar fluid, cavity rinse or swab, or organ rinse or swab sample. In some embodiments, the second samples is obtained at least about 1 hour after the first sample is obtained, e.g., about 1-18 hours after, about 1 day after, about 2 days after, about 3 days after, about 4 days after, about 5 days after, about 6 days after, about 7 days after, about 8 days after, about 9 days after, about 10 days after, about 2 weeks after, about 3 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after the first sample is obtained.

In some embodiments, the intervening agent is capable of stimulating release of one or more diseased cells, disease-associated exosomes, disease-associated transcriptomes, or disease-associated polynucleotides into circulation, optionally wherein the one or more diseased cells, disease-associated exosomes, disease-associated transcriptomes, or disease-associated polynucleotides are one or more tumor cells, tumor exosomes, tumor transcriptomes, or tumor DNA. In some embodiments, the intervening agent is a mobilizing agent, an anticancer agent, a chemotherapeutic agent, a monoclonal antibody, or a nutrient, or a combination thereof. In some embodiments, the intervening agent is one or more cytokines, chemokines, or growth factors or coagulation system modulators. In some embodiments, the one or more cytokines, chemokines, or growth factors comprises erythropoietin or a variant or analog thereof, methoxy polyethylene glycol-epoetin beta, G-CSF, PEGylated G-CSF, GM-CSF, SCF, IL-3, KGF, plerixafor, a CXCR4 antagonist, a GROβ or a fragment or analog thereof, or a combination thereof. In some embodiments, the one or more coagulation system modulators comprises wherein the one or more coagulation system modulators comprises heparin or a derivative thereof, a direct oral anticoagulant (DOAC), a tissue plasminogen activator (tPA), a streptokinase, an urokinase, a plasminogen activator inhibitor-1 (PAI-1) modulator, or a combination thereof.

In some embodiments, the intervening agent comprises plerixafor. In some embodiments, a provided method comprises administering 0.1-0.4 mg/kg plerixafor or about 10-25 mg plerixafor to the subject. In some embodiments, a method provided herein comprises administering about 0.16 mg/kg, about 0.24 mg/kg, about 13 mg plerixafor, or about 20 mg plerixafor to the subject. In some embodiments, a method provided herein comprises administering plerixafor to the subject subcutaneously, intramuscularly, intravenously, or by inhalation. In some embodiments, a method provided herein comprises administering plerixafor to the subject daily for 1-4 days. In some embodiments, a method provided herein comprises administering plerixafor to the subject once prior to obtaining the second sample. In some embodiments, a method provided herein comprises administering plerixafor to the subject 4-96 hours prior to obtaining the second sample. In some embodiments, a method provided herein comprises administering plerixafor to the subject about 11 hours prior to obtaining the second sample.

In some embodiments, the intervening agent comprises an cancer therapeutic.

In some embodiments, the intervening agent comprises a CXCR4 antagonist or GROβ or a fragment or analog thereof. In some embodiments, a method provided herein comprises administering the CXCR4 antagonist or GROβ or a fragment or analog thereof to the subject stimulates release of one or more diseased cells, disease-associated exosomes, disease-associated transcriptomes, or disease-associated polynucleotide into circulation, optionally wherein the one or more diseased cells, disease-associated exosomes, disease-associated transcriptomes, or disease-associated polynucleotides is one or more tumor cells, tumor exosomes, tumor transcriptomes, or tumor DNA. In some embodiments, the CRCX4 antagonist is motixafortide, balixafortide, or YF-H-2015005. In some embodiments, the GROβ or a fragment or analog thereof is MGTA-145.

In some embodiments, the CXCR4 antagonist or GROβ or a fragment or analog thereof is co-administered with a cytokine, chemokine, or growth factor. In some embodiments, co-administering the CXCR4 antagonist or GROβ or a fragment or analog thereof and the cytokine, chemokine, or growth factor to the subject stimulates release of one or more diseased cells, disease-associated exosomes, disease-associated transcriptomes, or diseased polynucleotide into circulation, optionally wherein the one or more diseased cells, disease-associated exosomes, disease-associated transcriptomes, or diseased polynucleotides is one or more tumor cells, tumor exosomes, tumor transcriptomes, or tumor DNA. In some embodiments, the cytokine, chemokine, or growth factor is a CXCR4 antagonist. In some embodiments, the cytokine, chemokine, or growth factor is selected from erythropoietin, G-CSF, GM-CSF, SCF, IL-3, KGF, motixafortide, balixafortide, YF-H-2015005, and plerixafor. In some embodiments, the cytokine, chemokine, or growth factor is plerixafor. In some embodiments, the cytokine, chemokine, or growth factor is G-CSF.

In some embodiments, the GROβ or a fragment or analog thereof is MGTA-145, and wherein MGTA-145 is co-administered with a cytokine, chemokine, or growth factor, optionally wherein the cytokine, chemokine, or growth factor is plerixafor or G-CSF. In some embodiments, the GROβ or a fragment or analog thereof is MGTA-145, and wherein MGTA-145 is co-administered with plerixafor. In some embodiments, the subject has been administered or is administered 0.0075-0.3 mg/kg, 0.015-0.15 mg/kg, or 0.03-0.15 mg/kg MGTA-145. In some embodiments, the subject has been administered or is administered about 0.0075 mg/kg, about 0.015 mg/kg, about 0.03 mg/kg, about 0.075 mg/kg, about 0.15 mg/kg, or about 0.3 mg/kg MGTA-145. In some embodiments, the subject is co-administered 0.1-0.4 mg/kg plerixafor or 5-50 mg plerixafor. In some embodiments, the subject is co-administered about 0.16 mg/kg, about 0.24 mg/kg, about 13 mg, or about 20 mg plerixafor. In some embodiments, the subject is co-administered about 5 mg, about 10 mg, about 15 mg, or about 20 mg plerixafor. In some embodiments, MGTA-145 is administered intravenously. In some embodiments, plerixafor is administered subcutaneously, intramuscularly, intravenously, or by inhalation.

In some embodiments, the CXCR4 antagonist is motixafortide, and wherein motixafortide is co-administered with G-CSF. In some embodiments, the subject has been administered or is administered 0.5-2 mg/kg motixafortide. In some embodiments, the subject has been administered or is administered about 0.5 mg/kg, about 0.75 mg/kg, about 1.0 mg/kg, about 1.25 mg/kg, about 1.5 mg/kg, or about 2 mg/kg motixafortide. In some embodiments, the subject is co-administered 5-20 μg/kg G-CSF. In some embodiments, the subject is co-administered about 10 μg/kg G-CSF. In some embodiments, the subject is co-administered about 100 μg, about 200 μg, about 250 μg, about 300 μg, about 480 μg, about 500 μg, about 1,000 μg G-CSF. In some embodiments, motixafortide is administered subcutaneously. In some embodiments, G-CSF is administered subcutaneously.

In some embodiments, the subject has or is suspected to have a cancer or cancer recurrence. In some embodiments, the subject does not have a cancer or cancer recurrence. In some embodiments, the subject does not have a cancer or cancer recurrence. In some embodiments, the subject has or is suspected to have a neurological condition, e.g., Alzheimer's disease or Parkinson's disease.

In some embodiments, the one or more diseased cells, disease-associated exosomes, disease-associated transcriptomes, or disease-associated polynucleotides are non-tumor-derived. In some embodiments, the one or more diseased cells, disease-associated exosomes, disease-associated transcriptomes, or disease-associated polynucleotides are one or more bacterial cells, bacterial exosomes, bacterial transcriptomes, or bacterial DNA. In some embodiments, the one or more diseased cells, disease-associated exosomes, disease-associated transcriptomes, or disease-associated polynucleotides is one or more tumor cells, tumor exosomes, tumor transcriptomes, or tumor DNA.

In some embodiments, provided herein is a method of detecting the presence or absence of minimal residual disease in a subject in need thereof, comprising determining presence or absent of one or more tumor cells, tumor exosomes, tumor transcriptomes, or tumor DNA, according to a method disclosed herein, wherein: presence of the one or more tumor cells, tumor exosomes, tumor transcriptomes, or tumor DNA in the fluid sample indicates presence of minimal residual disease in the subject, and absence of the one or more tumor cells, tumor exosomes, tumor transcriptomes, or tumor DNA in the fluid sample indicates absence of minimal residual disease in the subject.

In another aspect, provided herein is a method of treating cancer in a subject in need thereof, comprising administering at least one cancer therapeutic to the subject if one or more tumor cells, tumor exosomes, tumor transcriptomes, or tumor DNA has been detected in a fluid sample obtained from the subject following administration of an effective amount of a CXCR4 antagonist, GROβ or a fragment or analog thereof, or coagulation system modulator. In another aspect, provided herein is a method of treating cancer in a subject in need thereof, comprising: a. administering to the subject an effective amount of CXCR4 antagonist or GROβ or a fragment or analog thereof; b. obtaining a fluid sample from the subject; c. determining the presence or absence of one or more tumor cells, tumor exosomes, tumor transcriptomes, or tumor DNA in the fluid sample; and d. administering at least one cancer therapeutic to the subject if presence of one or more tumor cells, tumor exosomes, tumor transcriptomes, or tumor DNA in the fluid sample is detected.

In some embodiments, the cancer therapeutic is not an autologous HSC transplant.

In some embodiments, a cancer described herein is a primary cancer. In some embodiments, the cancer is solid tumor. In some embodiments, the cancer is selected from adrenal cancer, anal cancer, bile duct cancer, bladder cancer, bone cancer, cancer of the brain or central nervous system, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer (including head and neck squamous cell carcinoma), Hodgkin disease, Classical Hodgkin Lymphoma, diffuse large B cell lymphoma, follicular lymphoma, Kaposi sarcoma, kidney cancer, laryngeal and hypopharyngeal cancer, leukemia (including acute myeloid leukemia), liver cancer (including hepatocellular carcinoma), lymphoma, melanoma (including unresectable or metastatic melanoma), prostate cancer, lung cancer (including non-small cell lung cancer and metastatic non-small cell lung cancer) malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, renal cancer (including renal cell carcinoma), retinoblastoma, hematological malignancy, rhabdomyosarcoma, salivary gland cancer, sarcoma, squamous cell skin cancer, small intestine cancer, stomach cancer, testicular cancer, thymus cancer, thyroid cancer, uterine cancer, and vaginal cancer.

In some embodiments, the subject has received, is receiving, or will receive a cancer therapeutic. In some embodiments, the cancer therapeutic is immunotherapy (e.g., adoptive cell therapy, cancer vaccine, immunomodulator, oncolytic virus therapy, or targeted antibody), a chemotherapy, a radiation therapy, a hormone therapy, a stem cell transplant, or a combination thereof. In some embodiments, a sample is obtained around the time the subject is diagnosed a cancer or cancer recurrence or after the subject receives a cancer treatment, optionally after the subject completes one or more treatment cycles. In some embodiments, the sample is obtained after the subject completes one or more treatment cycles, optionally after the subject completes sufficient number of treatment cycles to stimulate a biomarker change in circulation.

In some embodiments, a method disclosed herein further comprises obtaining one or more samples from the subject and analyzing the biomarker.

In some embodiments, a cancer therapeutic of a method disclosed herein is selected from 5-fluorouracil, 6-mercaptopurine, 6-thioguanine, abemaciclib, abiraterone acetate, acalabrutinib, ado-trastuzumab emtansine, afatinib dimaleate, aldesleukin, alectinib, alemtuzumab, alpelisib, amifostine, aminolevulinic acid hydrochloride, anastrozole, apalutamide, arsenic trioxide, 1-asparaginase, atezolizumab, avelumab, axitinib, azacitidine, belinostat, bendamustine hydrochloride, bevacizumab, bexarotene, bicalutamide, binimetinib, bleomycin sulfate, blinatumomab, bortezomib, bosutinib, brentuximab vedotin, brigatinib, busulfan, cabazitaxel, cabozantinib-s-malate, calaspargase pegol-mknl, capecitabine, caplacizumab-yhdp, carboplatin, carfilzomib, carmustine, carmustine implant, cemiplimab-rwlc, ceritinib, cetuximab, chlorambucil, cisplatin, cladribine, clofarabine, cobimetinib, copanlisib hydrochloride, corticosteroids, crizotinib, cyclophosphamide, cytarabine, dabrafenib mesylate, dacarbazine, dacomitinib, dactinomycin, daratumumab, darolutamide, dasatinib, daunorubicin hydrochloride, daunorubicin hydrochloride and cytarabine liposome, decitabine, defibrotide sodium, degarelix, denileukin diftitox, denosumab, dexamethasone, dexamethasone, dexrazoxane hydrochloride, dinutuximab, docetaxel, doxorubicin hydrochloride, doxorubicin hydrochloride liposome, durvalumab, duvelisib, elotuzumab, eltrombopag olamine, emapalumab-lzsg, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, flutamide, fostamatinib disodium, fulvestrant, gefitinib, gemcitabine hydrochloride, gemtuzumab ozogamicin, gilteritinib fumarate, glasdegib maleate, glucarpidase, goserelin acetate, hydroxyurea, ibritumomab tiuxetan, ibrutinib, idarubicin hydrochloride, idelalisib, ifosfamide, imatinib mesylate, imiquimod, inotuzumab ozogamicin, interferon alfa-2b, recombinant, iobenguane I 131, ipilimumab, irinotecan hydrochloride, irinotecan hydrochloride liposome, ivosidenib, ixabepilone, ixazomib citrate, lanreotide acetate, lapatinib ditosylate, larotrectinib sulfate, lenalidomide, lenvatinib mesylate, letrozole, leuprolide acetate, lomustine, lorlatinib, mechlorethamine hydrochloride, megestrol acetate, melphalan, methotrexate, methylnaltrexone bromide, methylprednisolone, midostaurin, mitomycin c, mitoxantrone hydrochloride, mogamulizumab-kpkc, moxetumomab pasudotox-tdfk, necitumumab, nelarabine, neratinib maleate, netupitant and palonosetron hydrochloride, nilotinib, nilutamide, niraparib tosylate monohydrate, nivolumab, obinutuzumab, ofatumumab, olaparib, omacetaxine mepesuccinate, osimertinib mesylate, oxaliplatin, paclitaxel, paclitaxel albumin-stabilized nanoparticle formulation, palbociclib, palifermin, panitumumab, panobinostat, pazopanib hydrochloride, pegaspargase, peginterferon alfa-2b, pembrolizumab, pemetrexed disodium, pertuzumab, polatuzumab vedotin-piiq, pomalidomide, ponatinib hydrochloride, pralatrexate, prednisone, procarbazine hydrochloride, propranolol hydrochloride, raloxifene hydrochloride, ramucirumab, ravulizumab-cwvz, recombinant interferon alfa-2b, regorafenib, ribociclib, rituximab, rituximab and hyaluronidase human, rolapitant hydrochloride, romidepsin, romiplostim, rucaparib camsylate, ruxolitinib phosphate, selinexor, siltuximab, sonidegib, sorafenib tosylate, sunitinib malate, tagraxofusp-erzs, talazoparib tosylate, tamoxifen citrate, temozolomide, temsirolimus, thalidomide, thiotepa, tocilizumab, topotecan hydrochloride, toremifene, trabectedin, trametinib, trastuzumab, trastuzumab and hyaluronidase-oysk, trifluridine and tipiracil hydrochloride, uridine triacetate, valrubicin, vandetanib, vemurafenib, venetoclax, vinblastine sulfate, vincristine sulfate, vincristine sulfate liposome, vinorelbine tartrate, vismodegib, vorinostat, zanubrutinib, and ziv-aflibercept.

### BRIEF DESCRIPTION OF THE DRAWINGS

These and other features, aspects, and advantages of the present invention will become better understood with regard to the following description, and accompanying drawings, where:

FIG. 1 depicts the structure of plerixafor.

FIG. 2 depicts the structure of motixafortide.

FIG. 3 depicts the structure of balixafortide.

### DETAILED DESCRIPTION OF THE INVENTION

### Definitions

Terms used in the claims and specification are defined as set forth below unless otherwise specified.

As used in the specification and the appended claims, the singular forms “a,” “an” and “the” include plural referents unless the context clearly dictates otherwise.

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context.

As used herein, the term “actionable information” means any data that is may trigger an action, such as making a decision (e.g., treatment decision) or solving a problem (e.g., predicting treatment response).

The term “analog” or “analogue” refers to a mutant, variant, chimera, fusion, fragment, deletion, addition, or any other type of modifications made relative to a given polypeptide or protein (e.g., GROβ). In some embodiments, an “analog” of GROβ may be a GROβ fragment. In some embodiments, an “analog” of GROβ may have one or more such as 1, 2, 3, 4, 5, 6, 7, 8, 9, 10, 12, 14, 16, 18, 20, 22, 24, 26, 28, 30, 40, 50, 60, 70, 80 or more amino acid substitutions, deletions and/or insertions in comparison to the native sequence of GROβ, such as human GROβ as deposited with UniProt P19875. In some embodiments, an “analog” of GROβ may have an amino acid identity of at least 50%, 60%, 70%, 80%, 85%, 90% or 95% with a wild-type GROβ. In some embodiments, an “analog” of GROβ retains the biological activity of the wild-type GROβ, such as interacting with CXCR2.

As used herein, a “change” in connection to a given biomarker may be qualitative (e.g., presence or absence) or quantitative. The change may be any differences detectable by any methods known in the art. In some embodiments, a change, e.g., a change from absence to presence or a change in quantity, is indicative of increased disease risk.

The terms “co-administration” or “co-administer,” as used herein, refer to the administration of two or more agents, such that the two or more agents are administered as part of the same course of therapy. In some embodiments, two or more agents are co-administered when such agents are administered simultaneously. In some embodiments, the two or more agents are administered together, e.g., in the same formulation or, e.g., in different formulations but at the same time. In some embodiments, two or more agents are “co-administered” when such agents are administered separately, as long as the effects of the agents co-occur in the subject's body. In some embodiments, two or more agents are “co-administered” when such agents are administered separately, as long as one or more of the administered agents act to enhance or modulate the effect of the other administered agent(s). In some embodiments, two or more agents are “co-administered” when such agents are administered separately, as long as there is an overlap of an effect of each agent on the subject. In some embodiments, the two or more agents are administered within 24 hours (e.g., 12, 6, 5, 4, 3, 2, or 1 hour(s)) of one another, or within about 60, 30, 15, 10, 5, or 1 minute(s) of one another.

The term “detectable cancer,” as used herein, refers to any tumor materials (e.g., tumor cells, tumor exosomes, tumor transcriptomes, or tumor DNA) detectable by any conventional methodology available to one skilled in the art. In some embodiments, the term “has no detectable cancer,” when used in connection with a subject (e.g., patient), refers to a subject in which there is no detectable cancer by conventional methods, such as biopsy, urine or blood test, and imaging test (e.g., CT scan, magnetic resonance imaging (MM), ultrasound, or X-ray). In some embodiments, the term “has no detectable cancer” refers to a subject (e.g., patient) diagnosed with no cancer or cancer recurrence. In some embodiments, the term “no detectable cancer” refers to a situation where there is no detectable cancer by conventional liquid biopsy methods.

The term “diseased polynucleotides” as used herein refers to polynucleotides from a diseased cell, e.g., a tumor or cancer cell, or polynucleotides comprising a genetic profile, e.g., one or more genetic abnormalities associated with the disease.

The term “omics” as used herein refers a field of study in biology, encompassing any and all fields of study in biology ending with “omics,” such as genomics, metagenomic, transcriptomics, proteomics, and metabolomics. The term “omics profiling” or “omics analysis” as used herein refers to any process that yields omics information, including, but not limited to, genomics information, metagenomics information, epigenomics information, transcriptomics information, proteomics information, and metabolomics information. In some embodiments, the information may be a change in patterns of omics data.

The term “subject” as used herein includes both humans and non-humans and include but is not limited to humans, non-human primates, canines, felines, murines, bovines, equines, and porcines. In particular embodiments, the subject is a human subject.

The terms “polynucleotides,” “nucleic acid,” and “nucleotides” are used interchangeably to refer to a polymeric form of nucleotides of any length (e.g., 2 or more, 3 or more, 4 or more, 5 or more, 10 or more, 20 or more, 30 or more, 50 or more, 100 or more, 200 or more, 500 or more nucleotides in length, or even longer), either deoxyribonucleotides or ribonucleotides, or analogs thereof. The nucleic acids can be RNA, DNA, e.g., genomic DNA, mitochondrial DNA, viral DNA, synthetic DNA, or cDNA reverse transcribed from RNA.

The term “sufficient amount” or “effective amount” of an agent (e.g., any of the forgoing mobilizing agent), as used herein, is an amount sufficient to produce a desired effect, e.g., an amount sufficient to mobilize diseased cells, disease-associated exosomes, disease-associated transcriptomes, or diseased polynucleotides into circulation, enhancing or modulating the effect of the other administered agent, or to modulate/uncover biological characteristics not otherwise known. As such, an “effective amount” depends upon the context in which it is being applied. For example, in liquid biopsy applications, an “effective amount” may be an amount of an agent, alone or in combination with one or more other agent, sufficient to mobilize diseased cells, disease-associated exosomes, disease-associated transcriptomes, or diseased polynucleotides into circulation.

The term “therapeutically effective amount” is an amount that is effective to ameliorate a symptom of a disease. A therapeutically effective amount can be a “prophylactically effective amount” as prophylaxis can be considered therapy.

### Overview

Provided herein are methods, kits, and systems for liquid biopsy that enhance the yield (quantitative and qualitative) of diseased cells, disease-associated exosomes, disease-associated transcriptomes, diseased circulating nucleic acids, or combinations thereof in a liquid biological sample obtained from a subject. Such methods, kits, and systems utilize one or more mobilizing agents that mobilize diseased cells or their debris or disease-associated exosomes/transcriptomes from their niches into circulation of the subject, thereby enhancing yield of the liquid biopsy. The mobilized release of the one or more diseased cells, diseased-associated exosomes, disease-associated transcriptomes, or disease-associated polynucleotides into circulation, and subsequent collection of a fluid sample comprising such material, is referred to herein as liquid biopsy yield enhancement (LBYE). The mobilized release of the one or more diseased cells, diseased-associated exosomes, disease-associated transcriptomes, or disease-associated polynucleotides into circulation, and subsequent collection of a fluid sample comprising such material for biomarker analysis (e.g., determining presence or absence or change), is referred to herein as liquid biopsy yield enhancement plus (LBYE+). The collection of a fluid sample without mobilized release of the one or more diseased cells, disease-associated exosomes, disease-associated transcriptomes, or disease-associated polynucleotides into circulation and biomarker analysis is referred to herein as conventional liquid biopsy (cLB).

Also provided herein are methods, kits, and systems that for liquid biopsy that enhance the yield of clinically and/or therapeutically useful information (“actionable information”) obtained from a subject having a cancer. Such methods, kits, and systems compares data of biomarkers (e.g., hormones, proteins, genes, gene mutations, genetic amplifications or translocations, mutational or genetic profiles, genome-wide fragmentation profiles (“fragmentomes”), variant allele frequency (VAF), tumor mutational burden (TMB), microsatellite instability (MSI), telomere length, thymidylate synthase expression, DNA methylation pattern, or dysbiosis) obtainable from LBYE+ to that obtainable from cLB. The comparison data are useful for predicting treatment response and/or supporting treatment decision (including making a treatment decision that the previous liquid biopsy taught/guided away from).

“Release” of diseased cells or their debris or disease-associated exosomes/transcriptomes “into circulation” is used herein to refer to release of the cells or debris (e.g., diseased polynucleotides) or disease-associated exosomes/transcriptomes into a fluid compartment of the subject, such that the diseased cells or debris or disease-associated exosomes/transcriptomes can be obtained and/or detected in a fluid sample obtained from the subject. In some embodiments, the release of the diseased cells or their debris or disease-associated exosomes/transcriptomes into circulation comprises release into the bloodstream of the subject, such that they can be obtained and/or collected in a blood sample obtained from the subject. In some embodiments, the release of the diseased cells or their debris or disease-associated exosomes/transcriptomes into circulation comprises release into an extracellular fluid compartment, such that they can be obtained and/or collected in another type of fluid sample obtained from the subject. Exemplary fluid samples are disclosed herein. It is to be understood that the diseased cells, disease-associated exosomes, disease-associated transcriptomes, and/or diseased polynucleotides can be released into circulation from any organ or tissue of the body. In some embodiments, the diseased cells, disease-associated exosomes, disease-associated transcriptomes, and/or diseased polynucleotides are released into circulation from the subject's tumor. In some embodiments, the diseased cells, disease-associated exosomes, disease-associated transcriptomes, and/or diseased polynucleotides are released into circulation from the subject's bone marrow.

In some embodiments, the mobilizing agents comprise one or more cytokines or growth factors.

Accordingly, provided herein is a method of analyzing a fluid sample obtained from a subject, comprising determining presence or absence of one or more diseased cells, disease-associated exosomes, disease-associated transcriptomes, or disease-associated polynucleotides in the fluid sample, wherein the subject was previously administered one or more mobilizing agents in an amount effective to mobilize release of the one or more diseased cells, disease-associated exosomes, disease-associated transcriptomes, or disease-associated polynucleotides into circulation.

Also provided herein is a method of analyzing a fluid sample obtained from a subject, comprising analyzing biomarkers (e.g., hormones, proteins, genes, gene mutations, genetic amplifications or translocations, mutational or genetic profiles, TMB, MSI, telomere length, thymidylate synthase expression, DNA fragments, DNA methylation patterns, and/or dysbiosis) in the fluid sample, wherein the subject was previously administered one or more intervening agents (e.g., a mobilizing agent), optionally in an amount effective to mobilize release of one or more diseased cells, disease-associated exosomes, disease-associated transcriptomes, or disease-associated polynucleotides and/or modify their biological characteristics that can be detected/measured by any available assay into circulation.

Also provided herein is a method of detecting one or more diseased cells, disease-associated exosomes, disease-associated transcriptomes, or disease-associated polynucleotides in a fluid sample obtained from a subject, comprising: (a) administering to the subject one or more cytokines or growth factors in an amount effective to stimulate release of the one or more diseased cells (e.g., tumor cells), disease-associated exosomes (e.g., tumor exosomes), disease-associated transcriptomes (e.g., tumor transcriptomes), or disease-associated polynucleotides (e.g., tumor DNA) into circulation; (b) obtaining the fluid sample from the subject after administering the cytokine or growth factor to the subject; and (c) determining presence or absence of the released one or more diseased cells, disease-associated exosomes, disease-associated transcriptomes, or disease-associated polynucleotides in the fluid sample.

Also provided herein is a method of analyzing a biomarker in a subject, comprising the steps of: (a) obtaining a first sample from the subject and analyzing the biomarker; (b) administering to the subject one or more intervening agents; (c) obtaining a second sample from the subject and analyzing the biomarker; and (d) comparing the biomarker in the first and the second samples, thereby analyzing the biomarker. The comparison could be qualitative or quantitative in a manner that could influence choice of therapy.

Also provided herein is a method of predicting treatment response of a subject, comprising the steps of: (a) obtaining a first sample from the subject and analyzing the biomarker; (b) administering to the subject one or more intervening agents; (c) obtaining a second sample from the subject and analyzing the biomarker; and (d) determining change of the biomarker in the first and the second samples, thereby predicting treatment response by evaluating the change of the biomarker.

Also provided herein is a method of supporting treatment decision of a subject, comprising the steps of: (a) obtaining a first sample from the subject and analyzing the biomarker; (b) administering to the subject one or more intervening agents; (c) obtaining a second sample from the subject and analyzing the biomarker; and (d) determining change of the biomarker in the first and the second samples, thereby making an optimal treatment decision by evaluating the change of the biomarker.

Exemplary Intervening Agents

In some embodiments, the one or more intervening agents may be mobilizing agents, chemotherapeutic agents, monoclonal antibodies, anticancer agents, nutrients (e.g., folate, vitamin B12, or vitamin D), coagulation system modulators or any agents that stimulate molecular/DNA changes in a subject (e.g., stimulating release of tumor cells, tumor exosomes, tumor transcriptomes, or tumor DNA into circulation). A physician may determine the dosage regimen of the intervening agent. For example, in some embodiments, the intervening agent may be administered according to fixed dosing, weight-based dosing, or body surface area-based dosing.

Exemplary Coagulation System Modulators

In some embodiments, the coagulation system modulator may be heparin or a derivative thereof, a tissue plasminogen activator (tPA), a streptokinase, an urokinase, a direct oral anticoagulant (DOAC), or a plasminogen activator inhinbitor-1 (PAI-1) modulator. In some embodiments, the tPA is alteplase (Activase®). In some embodiments, exemplary DOACs include, but are not limited to, apixaban (Eliquis®), dabigatran (Pradaxa®), rivaroxaban (Xarelto®), edoxaban (Savaysa®), and betrixaban (Bevyxxa®).

Exemplary Mobilizing Agents

In some embodiments, the mobilizing agents comprise one or more cytokines or growth factors.

In some embodiments, the cytokine or growth factor is a CXCR4 antagonist. Exemplary CXCR4 antagonists include, but are not limited to, plerixafor, motixafortide, balixafortide, TG-0054, AMD070, FC122, FC131. Exemplary CXCR4 antagonists are described in Debnath et al. 2013 and Tsou et al. 2018.

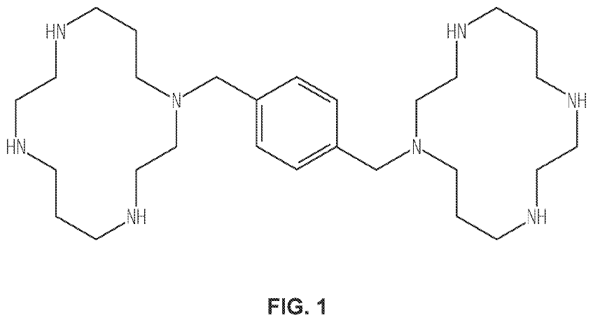

In some embodiment, the CXCR4 antagonist is plerixafor. The IUPAC name for plerixafor is 1-{[4-(1,4,8,11-tetrazacyclotetradec-1-ylmethyl)phenyl]methyl}-1,4,8,11-tetrazacyclotetradecane). The chemical structure of plerixafor is shown in FIG. 1. Plerixafor is an FDA approved treatment, used to mobilize HSC into circulation for collection and autologous transplant for the treatment of multiple myeloma and non-Hodgkin's lymphoma. Plerixafor has been shown to rapidly mobilize HSC within hours.

In some embodiments, the plerixafor is administered to the subject subcutaneously. In some embodiments, the plerixafor is administered to the subject intramuscularly. In some embodiments, the plerixafor is administered to the subject intravenously. In some embodiments, the plerixafor is administered to the subject by inhalation. In preferred embodiments, the plerixafor is administered to the subject subcutaneously.

In some embodiments, the plerixafor is administered to the subject once daily. In some embodiments, the plerixafor is administered to the subject once daily for 1-10 days, 1-8 days, 1-6 days, or preferably 1-4 days. In some embodiments, the plerixafor is administered once daily for one day. In some embodiments, the plerixafor is administered once daily for two days, three days, or four days.

In some embodiments, 0.1-0.4 mg/kg plerixafor is administered to the subject. In some embodiments, 0.24 mg/kg plerixafor is administered to the subject. In some embodiments, 0.16 mg/kg plerixafor is administered to the subject. For example, if the subject has an estimated creatinine clearance of less than 50 ml/min, the subject may be administered 0.16 mg/kg of the plerixafor. Preferably, the plerixafor is administered subcutaneously.

In some embodiments, 20 mg plerixafor is administered to the subject. In some embodiments, 13 mg plerixafor is administered to the subject. For example, if the subject has an estimated creatinine clearance of less than 50 ml/min, the subject may be administered 13 mg plerixafor. In some embodiments, 10 mg plerixafor is administered to the subject.

In some embodiments, the plerixafor is administered 48 hours or less prior to collection of the fluid sample from the subject. In some embodiments, the plerixafor is administered 36 hours or less prior to collection of the fluid sample from the subject. In some embodiments, the plerixafor is administered 24 hours or less prior to collection of the fluid sample from the subject. In some embodiments, the plerixafor is administered 20 hours or less prior to collection of the fluid sample from the subject. In some embodiments, the plerixafor is administered 16 hours or less prior to collection of the fluid sample from the subject. In some embodiments, the plerixafor is administered 12 hours or less prior to collection of the fluid sample from the subject. In some embodiments, the plerixafor is administered about 11 hours prior to collection of the fluid sample from the subject. In some embodiments, the plerixafor is administered about 6-48 hours prior to collection of the fluid sample from the subject. In some embodiments, the plerixafor is administered about 8-24 hours prior to collection of the fluid sample from the subject. In some embodiments, the plerixafor is administered about 10-14 hours prior to collection of the fluid sample from the subject.

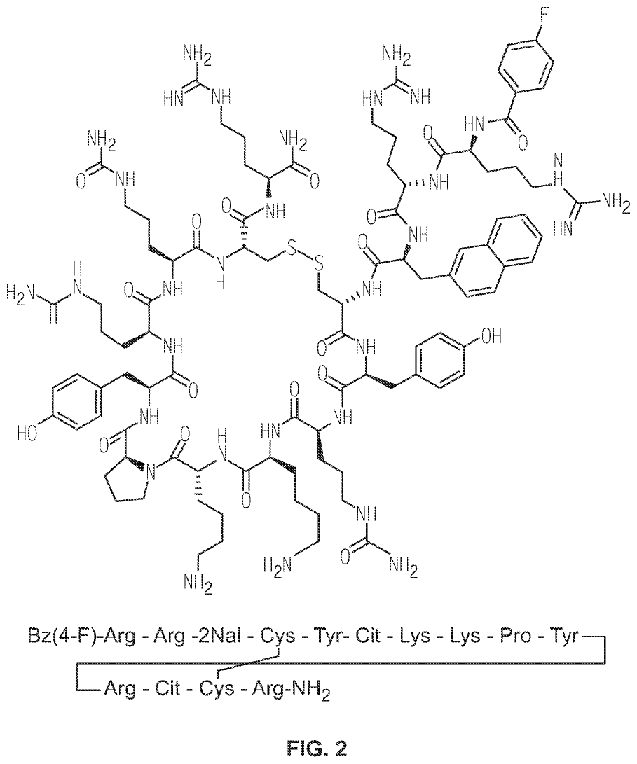

In some embodiments, the CXCR4 antagonist is motixafortide. Motixafortide is also known as BL-8040, BKT140, or TF14016. The chemical structure of motixafortide is shown in FIG. 2.

In some embodiments, 0.1-3 mg/kg motixafortide is administered to the subject. In some embodiments, 0.5-2 mg/kg motixafortide is administered to the subject. In some embodiments, about 0.5 mg/kg motixafortide is administered to the subject. In some embodiments, about 0.75 mg/kg of motixafortide is administered to the subject. In some embodiments, about 1.0 mg/kg of motixafortide is administered to the subject. In some embodiments, about 1.25 mg/kg of motixafortide is administered to the subject. In some embodiments, about 1.5 mg/kg of motixafortide is administered to the subject. In some embodiments, about 2.0 mg/kg of motixafortide is administered to the subject.

In some embodiments, motixafortide is administered to the subject once daily. In some embodiments, motixafortide is administered once daily for one day. In some embodiments, motixafortide is administered once daily for two days. In some embodiments, motixafortide is administered once daily for three days, once daily for four days, or once daily for five days. In some embodiments, motixafortide is administered to the subject once every two days, once every three days, once every four days, once every five days, once every six days, or once weekly.

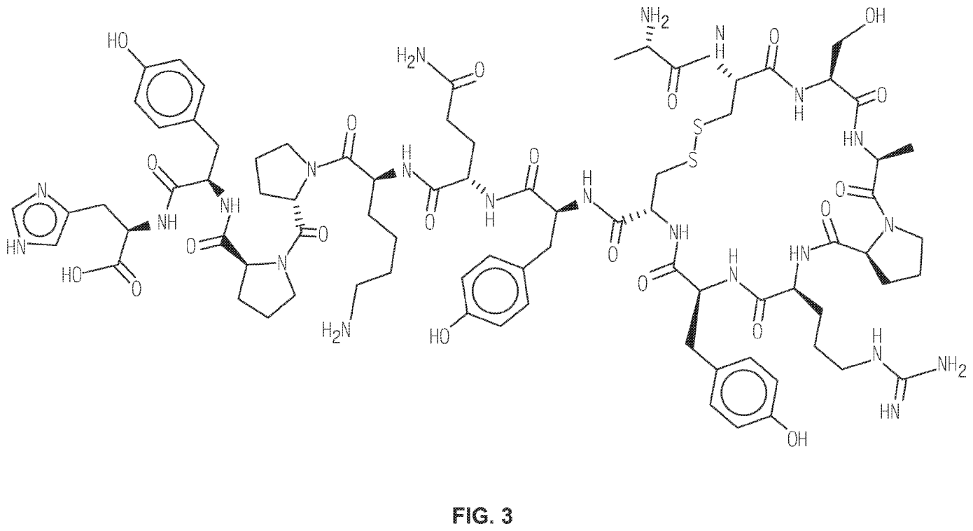

In some embodiments, the CXCR4 antagonist is balixafortide. Balixafortide is also known as POL6326. The chemical structure of balixafortide is shown in FIG. 3.

In some embodiments, 1-5.5 mg/kg balixafortide is administered to the subject. In some embodiments, about 1 mg/kg balixafortide is administered to the subject. In some embodiments, about 1.5 mg/kg of balixafortide is administered to the subject. In some embodiments, about 2 mg/kg of balixafortide is administered to the subject. In some embodiments, about 2.5 mg/kg of balixafortide is administered to the subject. In some embodiments, about 3 mg/kg of balixafortide is administered to the subject. In some embodiments, about 3.5 mg/kg of balixafortide is administered to the subject. In some embodiments, about 4 mg/kg of balixafortide is administered to the subject. In some embodiments, about 4.5 mg/kg of balixafortide is administered to the subject. In some embodiments, about 5 mg/kg of balixafortide is administered to the subject. In some embodiments, about 5.5 mg/kg of balixafortide is administered to the subject.

In some embodiments, balixafortide is administered to the subject once daily. In some embodiments, balixafortide is administered once daily for one day. In some embodiments, balixafortide is administered once daily for two days. In some embodiments, balixafortide is administered once daily for three days. In some embodiments, balixafortide is administered once daily for four days, once daily for five days, once daily for six days, or once daily for 1 week. In some embodiments, balixafortide is administered to the subject once every two days, once every three days, once every four days, once every five days, once every six days, or once weekly.

In some embodiments, the cytokine or growth factor is G-CSF. In some embodiments, 1-30 μg/kg G-CSF is administered to the subject. In some embodiments, 5-20 μg/kg G-CSF is administered to the subject. In some embodiments, about 10 micrograms/kg of G-CSF is administered to the subject. In some embodiments, about 300 μg is administered to a subject weighing 70 kg or less. In some embodiments, about 480 μg is administered to a subject weighing over 70 kg.

In some embodiments, G-CSF is administered to the subject once daily. In some embodiments, G-CSF is administered to the subject once daily for 1-10 days, 1-8 days, 1-6 days, or preferably 1-4 days. In some embodiments, G-CSF is administered once daily for one day. In some embodiments, G-CSF is administered once daily for two days, three days, or four days.

In some embodiments, G-CSF and plerixafor are co-administered to the subject. In some embodiments, G-CSF is administered to the subject prior to administration of plerixafor, such that G-CSF administration primes or enhances the mobilizing effects of the plerixafor on the diseased cells, exosomes, transcriptomes, or polynucleotides. In some embodiments, G-CSF is co-administered to the subject at the same time as the plerixafor or following plerixafor administration. In some embodiments, G-CSF and motixafortide are co-administered to the subject. In some embodiments, G-CSF is administered to the subject prior to the administration of motixafortide, such that G-CSF administration primes or enhances the mobilizing effects of motixafortide on the diseased cells, exosomes, transcriptomes, or polynucleotides. In some embodiments, G-CSF is co-administered to the subject at the same time as the motixafortide or following motixafortide administration.

In some embodiments of G-CSF and plerixafor co-administration, the G-CSF and plerixafor are co-administered to the subject once daily for one day. In another exemplary embodiment, the G-CSF and plerixafor are co-administered to the subject once daily for two days. In particular embodiments, about 5-20 μg/kg of G-CSF and about 0.1-0.4 mg/kg of plerixafor are co-administered to the subject. In particular embodiments, about and about 10 micrograms/kg of G-CSF and about 0.24 mg/kg plerixafor are co-administered to the subject. In particular embodiments, about 10 micrograms/kg of G-CSF and about 0.16 mg/kg plerixafor are co-administered to the subject. In some embodiments of G-CSF and motixafortide co-administration, the G-CSF and motixafortide are co-administered to the subject once daily for one day. In another exemplary embodiment, the G-CSF and motixafortide are co-administered to the subject once daily for two days. In particular embodiments, about 5-20 μg/kg of G-CSF and about 0.5-2 mg/kg of motixafortide are co-administered to the subject. In certain embodiments, about and about 10 μg/kg of G-CSF and about 1.25 mg/kg motixafortide are co-administered to the subject.

In an exemplary embodiment of G-CSF and plerixafor administration, G-CSF is administered to the subject once daily for 1-4 days at the dose of 5-20 μg/kg, optionally rounded off to the nearest vial size. In particular embodiments, 10 micrograms/kg of G-CSF is administered to the subject once daily for 1-4 days. Once the subject has received G-CSF once daily for 1-4 days, plerixafor administration is initiated. The plerixafor is administered once daily for 1-4 days. The subject's fluid sample is collected within 48 hours of the last plerixafor administration, e.g., around 11 hours after the last plerixafor administration. In an exemplary embodiment of G-CSF and motixafortide administration, G-CSF is administered to the subject once daily for 1-4 days at the dose of 5-20 μg/kg. In certain embodiments, 10 μg/kg of G-CSF is administered to the subject once daily for 1-4 days. In certain embodiments, after the subject has received G-CSF once daily for 1-4 days, motixafortide is administered once or twice separated by 1-3 days.

In an exemplary embodiment of G-CSF and plerixafor or motixafortide co-administration, G-CSF is administered to the subject once daily for one day, followed by commencement of plerixafor or motixafortide administration the following day. In an exemplary embodiment of G-CSF and plerixafor or motixafortide co-administration, G-CSF is administered to the subject once daily for two days, followed by commencement of plerixafor or motixafortide administration the following day. In an exemplary embodiment of G-CSF and plerixafor or motixafortide co-administration, G-CSF is administered to the subject once daily for three days, followed by commencement of plerixafor or motixafortide administration the following day. In an exemplary embodiment of G-CSF and plerixafor or motixafortide co-administration, G-CSF is administered to the subject once daily for four days, followed by commencement of plerixafor or motixafortide administration the following day.

In some embodiments, the cytokine or growth factor is GM-CSF. In some embodiments, 1-30 μg/kg GM-CSF is administered to the subject. In some embodiments, 5-20 μg/kg GM-CSF is administered to the subject. In some embodiments, about 10 micrograms/kg of GM-CSF is administered to the subject. In some embodiments, about 300 μg is administered to a subject weighing 70 kg or less. In some embodiments, about 480 μg is administered to a subject weighing over 70 kg.

In some embodiments, GM-CSF is administered to the subject once daily. In some embodiments, GM-CSF is administered to the subject once daily for 1-10 days, 1-8 days, 1-6 days, or preferably 1-4 days. In some embodiments, GM-CSF is administered once daily for one day. In some embodiments, GM-CSF is administered once daily for two days, three days, or four days.

In particular embodiments, GM-CSF and plerixafor are co-administered to the subject. In some embodiments, GM-CSF is administered to the subject prior to administration of plerixafor, such that GM-CSF administration primes or enhances the mobilizing effects of the plerixafor on the diseased cells, exosomes, transcriptomes, or polynucleotides. In some embodiments, GM-CSF is co-administered to the subject at the same time as the plerixafor or following plerixafor administration.

In some embodiments of GM-CSF and plerixafor co-administration, the GM-CSF and plerixafor are co-administered to the subject once daily for one day. In another exemplary embodiment, the GM-CSF and plerixafor are co-administered to the subject once daily for two days. In particular embodiments, about 5-20 μg/kg of GM-CSF and about 0.1-0.4 mg/kg of plerixafor are co-administered to the subject. In particular embodiments, about and about 10 micrograms/kg of GM-CSF and about 0.24 mg/kg plerixafor are co-administered to the subject. In particular embodiments, about 10 micrograms/kg of GM-CSF and about 0.16 mg/kg plerixafor are co-administered to the subject.

In an exemplary embodiment of GM-CSF and plerixafor administration, GM-CSF is administered to the subject once daily for 1-4 days at the dose of 5-20 μg/kg, optionally rounded off to the nearest vial size. In particular embodiments, 10 micrograms/kg of GM-CSF is administered to the subject once daily for 1-4 days. Once the subject has received GM-CSF once daily for 1-4 days, plerixafor administration is initiated. The plerixafor is administered once daily for 1-4 days. The subject's fluid sample is collected within 48 hours of the last plerixafor administration, e.g., around 11 hours after the last plerixafor administration.

In an exemplary embodiment of GM-CSF and plerixafor co-administration, GM-CSF is administered to the subject once daily for one day, followed by commencement of plerixafor administration the following day. In an exemplary embodiment of GM-CSF and plerixafor co-administration, GM-CSF is administered to the subject once daily for two days, followed by commencement of plerixafor administration the following day. In an exemplary embodiment of GM-CSF and plerixafor co-administration, GM-CSF is administered to the subject once daily for three days, followed by commencement of plerixafor administration the following day. In an exemplary embodiment of GM-CSF and plerixafor co-administration, GM-CSF is administered to the subject once daily for four days, followed by commencement of plerixafor administration the following day.

In some embodiments, the cytokine or growth factor is a CXCR2 agonist. Exemplary CXCR2 antagonists include, but are not limited to, CXCL1, CXCL2 (growth-related gene product (3, GROβ), CXCL3, CXCL5, CXCL6, CXCL7, and CXCL8, or a fragment thereof. In some embodiments, the cytokine or growth factor is GROβ or a fragment thereof. In some embodiments, the cytokine or growth factor comprises an amino acid sequence according to SEQ ID NO: 1 or a fragment thereof. In some embodiments, the cytokine or growth factor is MGTA-145. MGTA-145 is a truncated GROβ that activates the CXCR2 pathway in neutrophils (CXCR2 agonist).

In some embodiments, 0.0075-0.3 mg/kg MGTA-145 is administered to the subject. In some embodiments, 0.015-0.15 mg/kg MGTA-145 is administered to the subject. In some embodiments, 0.03-0.15 mg/kg MGTA-145 is administered to the subject. In some embodiments, about 0.0075 mg/kg MGTA-145 is administered to the subject. In some embodiments, about 0.015 mg/kg of MGTA-145 is administered to the subject. In some embodiments, about 0.03 mg/kg of MGTA-145 is administered to the subject. In some embodiments, about 0.075 mg/kg of MGTA-145 is administered to the subject. In some embodiments, about 0.15 mg/kg of MGTA-145 is administered to the subject. In some embodiments, about 0.3 mg/kg of MGTA-145 is administered to the subject.

In some embodiments, MGTA-145 is administered to the subject once daily. In some embodiments, MGTA-145 is administered once daily for one day. In some embodiments, MGTA-145 is administered to the subject once every two days, once every three days, once every four days, once every five days, once every six days, or once weekly.

In some embodiments, MGTA-145 and plerixafor are co-administered to the subject. In some embodiments, MGTA-145 is administered to the subject prior to administration of plerixafor. In some embodiments, MGTA-145 is administered to the subject at the same time as administration of plerixafor. In some embodiments, MGTA-145 is administered to the subject after administration of plerixafor. In some embodiments, MGTA-145 is administered to the subject immediately after, about 1 hour after, about 2 hour after, about 3 hour after, about 4 hour after, about 5 hour after, about 6 hour after, about 7 hour after, about 8 hour after, about 9 hour after, about 10 hour after, about 12 hour after, about 24 hour after, or about 48 hour after administration of plerixafor. In an exemplary embodiment of MGTA-145 and plerixafor co-administration, MGTA-145 is administered to the subject once daily for one day, following plerixafor administration. In an exemplary embodiment of MGTA-145 and plerixafor co-administration, MGTA-145 is administered to the subject once daily for one day, about 2 hours following plerixafor administration.

In some embodiments, about 0.0075-0.3 mg/kg MGTA-145 and about 0.1-0.4 mg/kg of plerixafor are co-administered to the subject. In some embodiments, about 0.015-0.15 mg/kg MGTA-145 and about 0.1-0.4 mg/kg of plerixafor are co-administered to the subject. In some embodiments, about 0.03-0.15 mg/kg MGTA-145 and about 0.1-0.4 mg/kg of plerixafor are co-administered to the subject. In some embodiments, about 0.0075 mg/kg MGTA-145 and about 0.24 mg/kg plerixafor are co-administered to the subject. In some embodiments, about 0.015 mg/kg MGTA-145 and about 0.24 mg/kg plerixafor are co-administered to the subject. In some embodiments, about 0.03 mg/kg MGTA-145 and about 0.24 mg/kg plerixafor are co-administered to the subject. In some embodiments, about 0.075 mg/kg MGTA-145 and about 0.24 mg/kg plerixafor are co-administered to the subject. In some embodiments, about 0.15 mg/kg MGTA-145 and about 0.24 mg/kg plerixafor are co-administered to the subject. In some embodiments, about 0.3 mg/kg MGTA-145 and about 0.24 mg/kg plerixafor are co-administered to the subject.

In some embodiments, the cytokine or growth factor is SCF. In some embodiments, the SCF is r-metHuSCF. In some embodiments, about 20 μg/kg r-metHuSCF is administered to the subject daily for 1-21 days. In some embodiments, about 20 μg/kg r-metHuSCF is administered to the subject daily for 1-10 days. In some embodiments, about 20 μg/kg r-metHuSCF is administered to the subject daily for 1-4 days, 1-3 days, 1-2 days, or for one day. In some embodiments, prior to the SCF treatment, the subject is pretreated with an anti-allergy medication, e.g., ranitidine. In some embodiments, about 20 μg/kg r-metHuSCF is co-administered to the subject with filgrastim. In some embodiments of co-administration of r-metHuSCF with filgrastim, about 1-20 μg/kg filgrastim is administered to the subject. In some embodiments of co-administration of r-metHuSCF with filgrastim, about 5-10 μg/kg filgrastim is administered to the subject.

In some embodiments, the cytokine or growth factor is IL-3. In some embodiments, about 5-10 μg/kg of IL-3 is administered to the subject.

In some embodiments, the cytokine or growth factor is erythropoietin. In some embodiments, about 10,000-40,000 U of erythropoietin is administered to the subject. In some embodiments, the cytokine or growth factor is a pegylated form of erythropoietin, such as Aranesp®. In some embodiments, about 100-400 μg of Aranesp® is administered to the subject.

In some embodiments, the cytokine or growth factor is KGF (keratinocyte growth factor, palifermin). In some embodiments, palifermin is administered at the dose of 10-100 mcg/kg per day for 1-3 days.

In some embodiments wherein the disease is cancer, the mobilizing agent is co-administered with an anticancer therapeutic. Any anticancer therapeutic known in the art may be co-administered with the mobilizing agent. In some embodiments, the anticancer therapeutic is a chemotherapeutic. In some embodiments, a combination of more than one anticancer therapeutic is used. Many chemotherapeutics are known in the art. Exemplary anti-cancer agents include, but are not limited to 5-Fluorouracil, 6-Mercaptopurine, 6-Thioguanine, Abemaciclib, Abiraterone Acetate, Acalabrutinib, Ado-Trastuzumab Emtansine, Afatinib Dimaleate, Aldesleukin, Alectinib, Alemtuzumab, Alpelisib, Amifostine, Aminolevulinic Acid Hydrochloride, Anastrozole, Apalutamide, Arsenic Trioxide, L-Asparaginase, Atezolizumab, Avelumab, Axitinib, Azacitidine, Belinostat, Bendamustine Hydrochloride, Bevacizumab, Bexarotene, Bicalutamide, Binimetinib, Bleomycin Sulfate, Blinatumomab, Bortezomib, Bosutinib, Brentuximab Vedotin, Brigatinib, Busulfan, Cabazitaxel, Cabozantinib-S-Malate, Calaspargase Pegol-mknl, Capecitabine, Caplacizumab-yhdp, Carboplatin, Carfilzomib, Carmustine, Carmustine Implant, Cemiplimab-rwlc, Ceritinib, Cetuximab, Chlorambucil, Cisplatin, Cladribine, Clofarabine, Cobimetinib, Copanlisib Hydrochloride, Corticosteroids, Crizotinib, Cyclophosphamide, Cytarabine, Dabrafenib Mesylate, Dacarbazine, Dacomitinib, Dactinomycin, Daratumumab, Darolutamide, Dasatinib, Daunorubicin Hydrochloride, Daunorubicin Hydrochloride and Cytarabine Liposome, Decitabine, Defibrotide Sodium, Degarelix, Denileukin Diftitox, Denosumab, Dexamethasone, Dexamethasone, Dexrazoxane Hydrochloride, Dinutuximab, Docetaxel, Doxorubicin Hydrochloride, Doxorubicin Hydrochloride Liposome, Durvalumab, Duvelisib, Elotuzumab, Eltrombopag Olamine, Emapalumab-lzsg, Enasidenib Mesylate, Encorafenib, Entrectinib, Enzalutamide, Epirubicin Hydrochloride, Erdafitinib, Eribulin Mesylate, Erlotinib Hydrochloride, Etoposide, Etoposide Phosphate, Everolimus, Exemestane, Fedratinib Hydrochloride, Fludarabine Phosphate, Flutamide, Fostamatinib Disodium, Fulvestrant, Gefitinib, Gemcitabine Hydrochloride, Gemtuzumab Ozogamicin, Gilteritinib Fumarate, Glasdegib Maleate, Glucarpidase, Goserelin Acetate, Hydroxyurea, Ibritumomab Tiuxetan, Ibrutinib, Idarubicin Hydrochloride, Idelalisib, Ifosfamide, Imatinib Mesylate, Imiquimod, Inotuzumab Ozogamicin, Interferon Alfa-2b, Recombinant, Iobenguane I 131, Ipilimumab, Irinotecan Hydrochloride, Irinotecan Hydrochloride Liposome, Ivosidenib, Ixabepilone, Ixazomib Citrate, Lanreotide Acetate, Lapatinib Ditosylate, Larotrectinib Sulfate, Lenalidomide, Lenvatinib Mesylate, Letrozole, Leuprolide Acetate, Lomustine, Lorlatinib, Mechlorethamine Hydrochloride, Megestrol Acetate, Melphalan, Methotrexate, Methylnaltrexone Bromide, Methylprednisolone, Midostaurin, Mitomycin C, Mitoxantrone Hydrochloride, Mogamulizumab-kpkc, Moxetumomab Pasudotox-tdfk, Necitumumab, Nelarabine, Neratinib Maleate, Netupitant and Palonosetron Hydrochloride, Nilotinib, Nilutamide, Niraparib Tosylate Monohydrate, Nivolumab, Obinutuzumab, Ofatumumab, Olaparib, Omacetaxine Mepesuccinate, Osimertinib Mesylate, Oxaliplatin, Paclitaxel, Paclitaxel Albumin-stabilized Nanoparticle Formulation, Palbociclib, Palifermin, Panitumumab, Panobinostat, Pazopanib Hydrochloride, Pegaspargase, Peginterferon Alfa-2b, Pembrolizumab, Pemetrexed Disodium, Pertuzumab, Polatuzumab Vedotin-piiq, Pomalidomide, Ponatinib Hydrochloride, Pralatrexate, Prednisone, Procarbazine Hydrochloride, Propranolol Hydrochloride, Raloxifene Hydrochloride, Ramucirumab, Ravulizumab-cwvz, Recombinant Interferon Alfa-2b, Regorafenib, Ribociclib, Rituximab, Rituximab and Hyaluronidase Human, Rolapitant Hydrochloride, Romidepsin, Romiplostim, Rucaparib Camsylate, Ruxolitinib Phosphate, Selinexor, Siltuximab, Sonidegib, Sorafenib Tosylate, Sunitinib Malate, Tagraxofusp-erzs, Talazoparib Tosylate, Tamoxifen Citrate, Temozolomide, Temsirolimus, Thalidomide, Thiotepa, Tocilizumab, Topotecan Hydrochloride, Toremifene, Trabectedin, Trametinib, Trastuzumab, Trastuzumab and Hyaluronidase-oysk, Trifluridine and Tipiracil Hydrochloride, Uridine Triacetate, Valrubicin, Vandetanib, Vemurafenib, Venetoclax, Vinblastine Sulfate, Vincristine Sulfate, Vincristine Sulfate Liposome, Vinorelbine Tartrate, Vismodegib, Vorinostat, Zanubrutinib, and Ziv-Aflibercept.

In some embodiments, the anticancer therapeutic is a therapeutic antibody, e.g., monoclonal antibody, or an antigen-binding fragment thereof. The therapeutic antibody or antigen-binding fragment thereof may be selective for an anti-tumor antigen, e.g., an antigen associated with the subject's cancer type or an antigen associated with the subject's tumor. In some embodiments, the therapeutic antibody or antigen-binding fragment thereof may be bispecific (e.g. bispecific T-cell engagers (BiTEs)) or polyspecific (i.e., capable of simultaneously binding to three or more types of antigens or epitopes).

In particular embodiments, wherein the subject is suspected to harbor residual disease but is in complete remission by conventional criteria, the cytokine or growth factor is co-administered with an appropriate dose of an anticancer therapeutic. The anticancer therapeutic may kill or inactivate any cancer cells that have been released into circulation, to prevent their engraftment at other sites. A physician may determine the appropriate anticancer therapeutic for inactivating the released cancer cells, while minimizing unnecessary side effects. A physician may determine the dosage regimen of the co-administered anticancer therapeutic which he or she considers appropriate for inactivating the released cancer cells, while minimizing unnecessary side effects. The physician may consider several factors in the determination, such as, e.g., the subject's medical history, the type of disease (e.g., type of cancer), the subject's age, body weight, gender, past response to therapeutic intervention, and the like. In some embodiments, about 1 μg-1 g of the anticancer therapeutic is co-administered with the plerixafor. In some embodiments, about 1 μg-1 g of the anticancer therapeutic is co-administered with the motixafortide. In some embodiments, about 1 μg-1 g of the anticancer therapeutic is co-administered with MGTA-145.

Exemplary Diseases

Methods and pharmaceutical compositions disclosed herein may be used in connection with any human diseases. In some embodiments, the disease is associated with a human organ(s), including but not limited to, liver, kidney, and lungs. In some embodiments, the disease is associated with liver. In some embodiments, the disease associated with liver may be autoimmune hepatitis (AIH), acute liver failure (ALF), alcoholic steatohepatitis (ASH), Hepatitis C, non-alcoholic fatty liver disease (NAFLD), non-alcoholic steatohepatitis (NASH), primary biliary cholangitis (PBC), or primary sclerosing cholangitis (PSC).

In some embodiments, the disease is non-malignant. In some embodiments, the disease is malignant.

In some embodiments, the disease is cancer. One or more LBYE methods described herein are useful for prognosis or minimal residual disease detection for any type of cancer. One or more LBYE+ methods described herein are useful for predicting treatment response and/or supporting treatment decision. Exemplary cancers include adrenal cancer, anal cancer, bile duct cancer, bladder cancer, bone cancer, cancer of the brain or central nervous system, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer (including head and neck squamous cell carcinoma), Hodgkin's disease, diffuse large B cell lymphoma, follicular lymphoma, Kaposi sarcoma, kidney cancer, laryngeal and hypopharyngeal cancer, leukemia (including acute and chronic leukemia involving the lymphoid, myeloid, both or unclassified lineages), liver cancer (including hepatocellular carcinoma), lymphoma, melanoma (including unresectable or metastatic melanoma), prostate cancer, lung cancer (including non-small cell lung cancer and metastatic non-small cell lung cancer), malignant mesothelioma, merkel cell carcinoma, metastatic urothelial carcinoma, multiple myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, renal cancer (including renal cell carcinoma), retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, or vaginal cancer.

In some embodiments, the cancer is associated with CXCR4 expression. Exemplary cancers associated with CXCR4 expression are described in Zhao, Hongli et al. 2015. Such cancers include, but are not limited to hematological malignancy, breast cancer, colorectal cancer, esophageal cancer, head and neck cancer, renal cancer, lung cancer, gynecologic cancer, liver cancer, prostate cancer and gallbladder cancer. Hematological malignancies include, e.g., multiple myeloma, Hodgkin's disease, non-Hodgkin lymphoma, acute leukemia, chronic leukemia, and myelodysplastic syndrome.

In particular embodiments, the cancer is multiple myeloma.

In some embodiments, the disease is a neurological condition. In some embodiments, the neurological condition is epilepsy, Alzheimer's disease or other types of dementia, migraines, strokes, Parkinson's disease, multiple sclerosis, or cerebral palsy. In some embodiments, the neurological condition is Alzheimer's disease or Parkinson's disease.

Exemplary Samples and Sample Processing

In some embodiments, the sample from the subject is a fluid sample. In some embodiments, the fluid sample is a whole blood sample. In some embodiments, the fluid sample is a plasma or serum sample. In some embodiments, the fluid sample is an ascites, cerebrospinal fluid, sweat, urine, tears, saliva, pleural fluid, pericardial fluid, lymph, cavity rinse, or organ rinse sample. The liquid sample can be an essentially cell-free liquid sample (e.g., plasma, serum, sweat, urine, tears, etc.). In some embodiments, the liquid sample is not essentially cell-free.

In some embodiments, a whole blood sample is obtained from the subject. The whole blood sample may be separated into fractions, e.g., cellular and non-cellular fractions. The cellular fraction may be assessed for the presence or absence of one or more CTCs. The non-cellular fraction (e.g., plasma or serum) may be assessed for the presence or absence of ctDNA or tumor exosomes/transcriptomes.

In some embodiments, a whole blood sample is separated into fractions by differential centrifugation. In an exemplary embodiment, a whole blood sample is separated into fractions using a Ficoll reagent (e.g., Ficoll-Paque PLUS, GE Healthcare). In some embodiments, a blood sample combined with Ficoll is subjected to density based centrifugation, resulting in splitting of the components into four distinct layers: (1) a red blood cell layer, (2) a Ficoll layer, (3) a mononuclear layer which contains white blood cells and other nucleated cells (e.g. CTCs) and (4) a plasma layer.

In some embodiments, one or more enrichment steps is performed on a cellular fraction of a blood sample, or a whole blood sample to enrich for CTCs. A skilled artisan may utilize any CTC enrichment process known in the art, including but not limited to those that enrich for CTCs by separating CTCs from other cells found in the blood. CTCs may be separated from other cells by physical properties, such as, e.g., size, density, electrical charge, and deformability. CTCs may be separated from other cells by biological properties, e.g., by positive or negative selection based on biomarker profile. Biomarker detection reagents, e.g., antibodies, may be selected based on the subject's tumor type and tumor profile. Exemplary methods for CTC enrichment are described in Harouaka, Ramdane A et al. 2013.

An exemplary system for CTC enrichment includes the EasySep™ Direct Human CTC Enrichment (StemCell Technologies), and CellSearch® (Menarini Silicon Biosystems).

In some embodiments, the sample from the subject is a tissue sample. In some embodiments, the tissue sample may be a cancerous tissue sample. In some embodiments, a tissue sample from the subject may be used as a source of cells, a source of RNA, a source of protein, or a source of thin sections, e.g., using immunohistochemistry (IHC) or flow cytometry. The tissue sample may be obtained using conventional biopsy instruments and procedures, such as needle biopsy, aspiration biopsy, CT-guided biopsy, ultrasound-guided biopsy, bone biopsy, bone marrow biopsy, liver biopsy, kidney biopsy, prostate biopsy, skin biopsy, or surgical biopsy. The tissue sample may be in any form sufficient for cell sorting, RNA extraction, protein extraction, or preparation of thin sections. Accordingly, the tissue sample may be fresh, preserved through suitable cryogenic techniques, or preserved through non-cryogenic techniques. An exemplary standard process for handling clinical biopsy specimens is to fix the tissue sample in formalin and then embed it in paraffin. Samples in this form are commonly known as formalin-fixed, paraffin-embedded (FFPE) tissue. Suitable techniques of tissue preparation for subsequent analysis are well-known to those of skill in the art.

Detection/Analysis of Diseased Cells, Disease-Associated Exosomes, Disease-Associated Transcriptomes, or Circulating Nucleic Acids (e.g., Disease-Associated Polynucleotides)

The presence of absence of diseased cells or material in the fluid sample may be determined by any means known in the art. The diseased cells or material may be tumor- or non-tumor-derived (e.g., immune).

In some embodiments, the diseased cells in the fluid sample are circulating tumor cells (CTCs). The CTCs may be from any one of the cancers disclosed herein. In some embodiments, the CTCs are from a cancer associated with CXCR4 expression. In some embodiments, the CTCs are from a hematological malignancy. In particular embodiments, the CTCs are from multiple myeloma. Exemplary cancers, cancers associated with CXCR4 expression, and hematological malignancies are disclosed herein.

In particular embodiments, the CTCs are detected using one or more biomarkers specific for the tumor.

In particular embodiments wherein the cancer is multiple myeloma, the one or more biomarkers are selected from CD138, CD38, CD45, CD56, CD19, cytoplasmic κ and λ immunoglobulin light chains, CD20, CD27, CD28, CD81, CD117, CD200, CD54, CD229, CD319, and VS38c. In particular embodiments wherein the cancer is multiple myeloma, the one or more biomarkers are selected from CD138, CD38, CD45, CD56, CD19, and cytoplasmic κ and λ immunoglobulin light chains. In particular embodiments wherein the cancer is multiple myeloma, the one or more biomarkers are selected from CD19, CD45, CD56, CD81, CD27, CD117, and cytoplasmic κ and λ immunoglobulin light chains. The one or more biomarkers may be positive or negative markers. In some embodiments, wherein the cancer is multiple myeloma, the CTCs are detected using multiparametric flow cytometry for the one or more biomarkers. In some embodiments, the multiparametric flow cytometry comprises gating for any one or more of CD138, CD38, CD45, CD56, CD19, cytoplasmic κ and λ immunoglobulin light chains, CD20, CD27, CD28, CD81, CD117, CD200, CD54, CD229, CD319, and VS38c. In particular embodiments, the multiparametric flow cytometry comprises gating for any one or more of CD138, CD38, CD45, CD56, CD19, and cytoplasmic κ and λ immunoglobulin light chains. In other particular embodiments, the multiparametric flow cytometry comprises gating for CD19, CD45, CD56, CD81, CD27, CD117, and cytoplasmic κ and λ immunoglobulin light chains. In preferred embodiments, the multiparametric flow cytometry comprises gating for CD138. In some embodiments, wherein the subject had been previously treated with an anticancer therapy comprising a monoclonal antibody against CD38 or CD138, the multiparametric flow cytometry comprises gating for any one or more of CD54, CD229, CD319, and VS38c. Exemplary multiparametric flow cytometry methods for detection of multiple myeloma are described herein, and in, e.g., Kumar S et al. 2016; WO2017198879A1; Flores-Montero J et al. 2017; Mishima Y et al. 2017; US20180140664A1.

In particular embodiments wherein the cancer is multiple myeloma, ASO-qPCR to may be used to detect presence or absence of multiple myeloma CTCs in the fluid sample. Exemplary ASO-qPCR techniques are described in Kumar S et al. 2016.

In some embodiments, the CTCs from the fluid sample are analyzed by sequencing, e.g., single cell sequencing. The sequencing can comprise whole exome sequencing, whole genome sequencing, targeted sequencing of a panel of cancer genes, or targeted sequencing of a single cancer gene. The sequencing can comprise next generation sequencing, e.g., as described herein. The sequencing can comprise deep sequencing, e.g., deep sequencing of the VDJ region.

In some embodiments, the CTCs may be analyzed for one or more cancer biomarkers using any methods known in the art. In some embodiments, the one or more cancer biomarkers are protein biomarkers, e.g., AFP, androgen receptor, CA15-3, CA19-9, CA125, CA27.29, calcitonin, CD44, CEA, cytokeratin 5/6, DNAPKcs, EGFR, estrogen receptor, FDP, ferritin, FOXA1, GATA3, HCGO, HER2, HE4, HP1f3, KIT, LAG-3, NMP-22, NSE, PD-1, P-REX1, progesterone receptor, OVAL osteocalcin, Pro2PSA, PSA, S100, SCC, thyroglobulin, transferrin receptor, and/or TPA. In some embodiments, the one or more cancer biomarkers are tumor-associated mutations,

In particular embodiments wherein the cancer is lymphoma, the one or more biomarkers are selected from CD4, CD5, CD8, CD10, CD19, CD20, CD22, CD23, CD30, CD38, and surface κ and λ immunoglobulin light chains.

In some embodiments, the biomarker is detected by antibody staining. In particular embodiments, cells that are positive for the biomarker are detected by flow cytometry.

The presence or absence of disease-associated exosomes/transcriptomes in the fluid sample may be determined by any means known in the art. Circulating exosomes/transcriptomes may be isolated from the sample using any means known in the art.

The presence or absence of diseased circulating nucleic acids in the fluid sample may be determined by any means known in the art.

In some embodiments, cell-free or circulating nucleic acids may be isolated from the fluid sample, e.g., a cell-free fluid sample. Nucleic acid can be isolated from the sample using any means known in the art. For example, nucleic acid can be extracted from the sample using liquid extraction (e.g., Trizol, DNAzol) techniques. Nucleic acid can also be extracted using commercially available kits (e.g., Qiagen DNeasy kit, QIAamp kit, Qiagen Midi kit, QIAprep spin kit).

Nucleic acid can be concentrated by known methods, including, by way of example only, centrifugation. Nucleic acid can be bound to a selective membrane (e.g., silica) for the purposes of purification. Nucleic acid can also be enriched for fragments of a desired length, e.g., fragments which are less than 1000, 500, 400, 300, 200 or 100 base pairs in length. Such an enrichment based on size can be performed using, e.g., PEG-induced precipitation, an electrophoretic gel or chromatography material, gel filtration chromatography, or TSK gel.

Polynucleotides extracted from a biological sample can be selectively precipitated or concentrated using any methods known in the art.

The nucleic acid sample can be enriched for target polynucleotides. Target enrichment can be by any means known in the art. For example, the nucleic acid sample may be enriched by amplifying target sequences using target-specific primers. The target amplification can occur in a digital PCR format, using any methods or systems known in the art. The nucleic acid sample may be enriched by capture of target sequences onto an array immobilized thereon target-selective oligonucleotides. The nucleic acid sample may be enriched by hybridizing to target-selective oligonucleotides free in solution or on a solid support. The oligonucleotides may comprise a capture moiety which enables capture by a capture reagent. Capture moiety/capture reagent pairs are known in the art. In some embodiments the capture reagent is avidin, streptavidin, or neutravidin and the capture moiety is biotin. In another embodiment the capture moiety/capture reagent pair is digoxigenin/wheat germ agglutinin.

In some embodiments, the nucleic acid sample is not enriched for target polynucleotides, e.g., represents a whole genome.

In some embodiments, circulating nucleic acids from the fluid sample may be analyzed for genetic abnormalities associated with disease using any methods known in the art. In some embodiments, diseased circulating polynucleotides from the fluid sample are detected by the presence or absence of one or more genetic abnormalities associated with the disease. For instance, ctDNA (circulating tumor DNA) may be detected based on presence or absence of one or more cancer-associated genetic abnormalities. Many types of genetic abnormalities are known in the art and may include mutations to one or more genes, mutations to a chromosome, and/or mutations to the genetic sequence. Mutations to many genes are known in art to be associated with cancer and may include mutations to ATM, BARD1, BRCA1, BRCA2, BRIP1, BAP1, BRCA2, CDH1, CDK4, CDKN2A, CHEK2, NF1, EPCAM, MLH1, MSH2, MSH6, MUTYH, NBN, PALB2, PMS2, PTEN, RAD51C, RAD51D, STK11, and/or TP53. Many types of chromosomal abnormalities are known in the art and may include a structural abnormality (e.g., translocations, inversions, or insertions) or an atypical number of chromosomes (e.g., copy number variations such as deletions or duplications).

In the case of multiple myeloma, the diseased circulating polynucleotides may comprise one or more mutations in one or more genes selected from KRAS, NRAS, TP53, DIS3, FAM46C, BRAF, TRAF3, PRDM1, CYLD, RB1, ACTG1, IRF4, IDH1, INTS12, SP140, LTB, MAX, HIST1H1E, EGR1, FGFR3, FNDC3A, TNKS, BCL7A, RPL10, GCET2, RASA2, PLA2G2D, C9orf80, HIST1H3G, CDKN1B, RNF151, C17orf77, FAM153B, SLC24A1, OR1L8, USP50, CXCR4, KRTDAP, FBXO36, ROBO1, TGDS, SNX7, MPEG1, DHX32, RYR2, NFKBIA, FSIP2, SI, NECAB3, COASY, EIF4G2, ZFHX4, CCND1, LRRC16A, YTHDF2, PHOX2B, C15orf59, MOGAT3, EXOG GRIA2, C4orf43, CCDC144NL, CKM, OR1N2, PRIM2, OR1S2, NDUFAF3, C20orf112, HIST1H3H, and PNRC1. In some cases a genetic abnormality is one or more of KRAS (p.G12D), KRAS (p.Q61H), NRAS (p.G12D), BRAF (p.G469R), IRF4 (p.L116R), SLC24A1 (p.R686G), MPEG1 (p.G537E), and RYR2 (p.I784V). Exemplary genetic abnormalities associated with multiple myeloma are described in US2018/0305766A1.

In some embodiments, circulating nucleic acids from the fluid sample may be analyzed for one or more cancer biomarkers using any methods known in the art. In some embodiments, the one or more cancer biomarkers may be associated with treatment response. In some embodiments, the treatment may comprise an immunotherapy (e.g., adoptive cell therapy, cancer vaccine, immunomodulator, oncolytic virus therapy, or targeted antibody), a chemotherapy, a radiation therapy, a hormone therapy, a stem cell transplant, or a combination thereof. In some embodiments, the one or more biomarkers may be selected from variant allele frequency (VAF), tumor mutational burden (TMB), microsatellite instability (MSI), altered genome-wide fragmentation profiles, DNA methylation pattern, and dysbiosis.

In some embodiments, the circulating nucleic acids (e.g., diseased circulating polynucleotides) from the fluid sample are detected by next generation sequencing. The next generation sequencing may comprise sequencing of immunoglobulin gene segments. Exemplary next generation sequencing techniques are described in Kumar S et al. 2016.

Kits

Also provided herein is a kit, comprising a pharmaceutically acceptable dosage form of a mobilizing agent disclosed herein, and instructions for use.

In some embodiments, such kits comprise a carrier, package or container that is compartmentalized to receive one or more containers such as vials, tubes, and the like, each of the container(s) comprising one of the separate elements to be used in the method. The kit can comprise the container described above and one or more other containers comprising materials desirable from a commercial end user standpoint, including buffers, diluents, filters, and package inserts with instructions for use. In addition, a label can be provided on the container to indicate that the composition is used for a specific therapeutic application and can also indicate directions for either in vivo or in vitro use, such as those described above. Directions and or other information can also be included on an insert which is included with the kit.

Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for, e.g., generating a report of a subject's MRD determination, prognosis, genetic profile of the one or more diseased cells, disease-associated exosomes, disease-associated transcriptomes, or diseased polynucleotides obtained by a method disclosed herein, and optionally transmitting the report over a network.

Also provided herein is a computer system comprising a computer readable medium disclosed herein.

Systems

Also provided herein is a system for LBYE or LBYE+. In some embodiments, the system comprises one or more pharmaceutically acceptable dosage forms of a mobilizing agent disclosed herein (e.g., one or more dosage forms of plerixafor, motixafortide, balixafortide, G-CSF or MGTA-145, or a combination thereof, for administration to the subject). The system may further comprise reagents, devices, and/or kits for obtaining a fluid sample from a subject in need thereof (e.g., a fluid collection tube and optionally reagents for enhancing stability of the fluid sample). The system may further comprise one or more reagents, devices, and/or apparatuses for analyzing diseased cells, disease-associated exosomes, disease-associated transcriptomes, or diseased polynucleotides (e.g., one or more reagents, devices, and/or apparatuses for multiparametric flow cytometry analysis, a sequencer, and the like).

Methods for MRD Detection

The methods described herein can be used for MRD detection in a subject. Such methods are particularly advantageous for the detection of MRD, because mobilization of diseased cells (e.g., tumor cells), debris from diseased cells (e.g., ctDNA), and/or disease-associated exosomes/transcriptomes, enhances yield of the diseased cells, disease-associated exosomes, disease-associated transcriptomes, and/or disease-associated polynucleotides, thus enhancing sensitivity of the assay and reducing false negatives. The methods described herein, when used for MRD detection, are advantageous compared to conventional MRD methods known in the art (e.g., the Altera or Signatera assays) and provide benefits and improvements, e.g., enhanced sensitivity, when used in combination with any conventional MRD methods. For example, Altera relates to a whole exome and transcriptome-based approach for genomic profiling of certain medically important genes and oncology biomarkers. LBYE, when used with the Altera, will enhance the assay's ability to identify abnormalities that are not otherwise detectable. Signatera relates to a personalized, tumor-informed assay, which detects ctDNA for MRD assessment and monitoring for relapse. LBYE, when used with the Signatera assay, will enhance the assay's ability to identify ctDNA that is otherwise undetectable.

Accordingly, in some embodiments, a detected presence of tumor cells, tumor exosomes, tumor transcriptomes, or ctDNA in the fluid sample is indicative of MRD in the subject. Likewise, detected absence of tumor cells, tumor exosomes, tumor transcriptomes, or ctDNA in the fluid sample is indicative of a true MRD absence in the subject (“LBYE CR” or “LBYE complete remission” or “enhanced liquid biopsy CR”). In some embodiments, the method further comprises administering a cancer therapeutic to the subject if MRD is detected.

Accordingly, provided herein is a method of detecting presence or absence of MRD in a subject in need thereof, comprising analyzing a fluid sample obtained from the subject or detecting one or more tumor cells, tumor exosomes, or tumor DNA in a fluid sample obtained from the subject, according to a method described herein, wherein (i) presence of the one or more tumor cells, tumor exosomes, tumor transcriptomes, or tumor DNA in the fluid sample indicates presence of MRD in the subject, and (ii) absence of the one or more tumor cells, tumor exosomes, tumor transcriptomes, or tumor DNA in the fluid sample indicates absence of MRD in the subject.

In some embodiments of an MRD detection method, administration of the mobilizing agent, subsequent fluid sample collection, and analysis of the fluid sample is performed when the subject is determined to be in remission or suspected by a clinician to be in complete remission from cancer. In some embodiments of an MRD detection method, administration of the mobilizing agent, subsequent fluid sample collection, and analysis of the fluid sample is performed after the subject has concluded a course of disease therapy, e.g., a course of anticancer therapy. In some embodiments of an MRD detection method, administration of the mobilizing agent, subsequent fluid sample collection, and analysis of the fluid sample is performed after the subject's cancer is undetectable by conventional means. In some embodiments, administration of the mobilizing agent, subsequent fluid sample collection, and analysis of the fluid sample is performed after the subject has completed sufficient anticancer therapy as to render to cancer undetectable by conventional means such as microscopy, measurement of tumor markers, standard flow cytometry, biopsy, imaging studies (e.g., X-rays, CT scans, radionuclide scans, PET scans, MRI scans). Conventional means may include means known in the art such as, e.g., medical imaging, cLB (liquid biopsy without enhancement by use of one or more mobilizing agents described herein), and solid tissue biopsy.

In some embodiments, presence of diseased cells, disease-associated exosomes, disease-associated transcriptomes, or diseased polynucleotides in the fluid sample obtained according to an LBYE method disclosed herein indicates that the subject has MRD. In some embodiments, absence of diseased cells, disease-associated exosomes, disease-associated transcriptomes, or diseased polynucleotides in the fluid sample obtained according to an LBYE method disclosed herein indicates lack of MRD in the subject, and provides a more accurate indication that the subject is in a true complete remission (“LBYE CR”). Methods for detecting presence or absence of diseased cells in the fluid sample are disclosed supra. Methods for detecting presence or absence of disease-associated exosomes/transcriptomes in the fluid sample are disclosed supra. Methods for detecting presence or absence of diseased polynucleotides in the fluid sample are disclosed supra.

In some embodiments, the administration of the one or more mobilizing agents, the subsequent collection of the fluid sample, and the analysis of the fluid sample is performed once. In some embodiments, the administration of the one or more mobilizing agents, the subsequent collection of the fluid sample, and the analysis of the fluid sample is performed more than once (i.e.—at least two times). For example, if the initial LBYE method detects presence of MRD in the subject, the subject may be administered an additional course (or more than one course) of anticancer therapy, and the LBYE method performed for detection of MRD after the subject has concluded the additional course(s) of therapy. For other example, if the initial LBYE method detects absence of MRD, the LBYE method may be performed one or more subsequent times, in order to monitor the subject for MRD resurgence or relapse. In some embodiments, wherein the initial LBYE test detects absence of MRD, maintenance therapy may be administered to the subject. In some embodiments, if initial LBYE method detects presence of MRD, the LBYE method may also be performed one or more subsequent times, in order to monitor the subject for MRD resurgence or relapse. For example, in some embodiments, an increase in MRD detected by an subsequently performed LBYE method indicates the resurgence or relapse. In some embodiments, wherein the initial LBYE test detects presence of MRD, maintenance therapy may be administered to the subject. Maintenance therapy generally comprises administration of an anticancer agent in an amount effective to maintain a therapeutic benefit to the subject that was achieved via a therapeutic anticancer regimen, e.g., (1) inhibiting an increase in the number of cancer cells; (2) inhibiting an increase in tumor size; (3) inhibiting cancer cell infiltration into peripheral organs; (4) inhibiting tumor metastases; (5) relieving or reducing to some extent one or more of the symptoms associated with the disorder; and/or (6) inhibiting recurrence or relapse of the cancer. In some embodiments, the maintenance therapy comprises administration of the therapeutic anticancer regimen at a lower dosage scheme. In some embodiments, wherein the initial LBYE method was negative for MRD, disease therapy for the subject is terminated. In some embodiments wherein the initial LBYE test detects presence of MRD, maintenance therapy or standard therapy is administered to the subject. In some embodiments, the LBYE method is repeated at regular intervals for the monitoring of MRD resurgence or relapse. In some embodiments, the LBYE method is performed at a frequency of about once a month, one every 2 months, once every 3 months, once every 4 months, once every 6 months, once a year.

In some embodiments, a report of the determination of a subject's MRD status according to any of the methods disclosed herein is transmitted over a network.

Determination of Complete Remission

The MRD detection methods disclosed herein are particularly useful in certain embodiments, such as detection of MRD when the subject has been determined to be, or suspected of being, in complete remission. Accordingly, in some embodiments, administration of the mobilizing agent, subsequent fluid sample collection, and analysis of the fluid sample is performed when the subject is determined or suspected of being in remission. In some embodiments, the subject is determined or suspected of being in remission from multiple myeloma.

In some embodiments, a subject with myeloma is determined or suspected to be in remission when the subject has completed a course of anticancer therapy. For example, a subject may be expected to be in remission when the subject has undergone an autologous stem cell transplant (ASCT). In some embodiments, the subject is expected to be in remission one month, two months, three months, or more than three months after undergoing ASCT. In some embodiments, the subject is suspected of being in remission about 100 days after receiving ASCT.

In some embodiments, administration of the mobilizing agent, subsequent fluid sample collection, and analysis of the fluid sample is performed when the subject is determined or suspected of being in remission from multiple myeloma according to conventional criteria.

A skilled artisan, e.g., a clinician, may determine whether a subject with myeloma is expected to be in remission according to any conventional criteria known in the art. Exemplary embodiments of conventional criteria for determining whether a subject with myeloma is expected to be in remission are described in Kumar et al. (Lancet Oncology). In some embodiments, a subject with myeloma is determined or expected to be in remission according to conventional criteria when all of following conditions (1)-(6) are met: (1) no abnormal (clonal) plasma cells in the bone marrow, (2) disappearance of the original disease-specific monoclonal protein from blood and/or urine on immunofixation electrophoresis, (3) disappearance of all plasmacytomas (tumors), (4) lack of new bone lesions on one or more imaging studies (X-rays, CT scans, MM scans, PET scan), (5) no disproportionate elevation of the involved (disease-specific) serum free light chain level, and (6) no concordant abnormal free light chain ratio in serum. For example, in some embodiments, disappearance of the original disease-specific monoclonal protein from blood and/or urine on immunofixation electrophoresis may be defined as remission according to conventional criteria. In some embodiments, a subject with myeloma is determined or expected to be in remission according to conventional criteria when the first two of conditions (1)-(6) are met. In some embodiments, a subject with myeloma is determined or expected to be in remission according to conventional criteria when the first three of conditions (1)-(6) are met. In some embodiments, a subject with myeloma is determined or expected to be in remission according to conventional criteria when the first four of conditions (1)-(6) are met. In some embodiments, a subject with myeloma is determined or expected to be in remission according to conventional criteria when the first five of conditions (1)-(6) are met. By way of example only, a subject with myeloma is determined or expected to be in remission according to conventional criteria when conditions (2)-(6) are met. In some embodiments, a subject with myeloma is determined or expected to be in remission according to conventional criteria when the second of conditions (1)-(6) is met (“serologic CR”). In the foregoing examples, the subject can be confirmed to be in complete remission when the LBYE method detects no CTC or ctDNA in the fluid sample of the subject. It is known that a minority of the malignant cell population in myeloma—usually the one most difficult to eliminate with therapy—does not secrete paraprotein (the abnormal protein easily detectable in the blood). LBYE is especially important to identify MRD in this situation—and can potentially change the current definition of CR in myeloma (LBYE CR).

Bone lesions may be detectable by medical imaging, according to any method known in the art. The medical imaging may comprise skeletal radiography (e.g., X-ray), CT (including low-dose whole body CT), MRI, ¹⁸F-fluorodeoxyglucose (FDG) PET, and FDG-PET with CT (PET-CT). In some embodiments, a bone lesion is diagnosed by the presence of one or more sites of osteolytic bone destruction (>5 mm in size) seen on CT (including low dose whole-body CT) or PET-CT.

Serum free light chain levels and ratios can be determined according to any method known in the art. The free light chain (FLC) assay measures the ratio of free κ and λ light immunoglobulin chains (unbound to immunoglobulin heavy chains) in the serum. The normal ratio for FLC-κ/λ is 0.26-1.65. Ratios outside the normal range, e.g., ratios of about 100 are used to indicate multiple myeloma. Such assays are described in Rajkumar S et al. 2014.

Abnormal (clonal) plasma cells in a bone marrow sample of the subject can be detected according to any methods known in the art.

In some embodiments, abnormal plasma cells of the bone marrow are detected by core bone marrow biopsy with immunohistochemical staining with CD138 antibody. In some embodiments, abnormal plasma cells of the bone marrow are detected by counting cells on a marrow aspirate smear.

In some embodiments, abnormal plasma cells of the bone marrow are detected by flow cytometry of a bone marrow sample of the subject. In some embodiments, multiple myeloma cells in the bone marrow sample are detected by flow cytometry. The flow cytometry may comprise multiparametric flow cytometry. Such techniques can be used to distinguish multiple myeloma plasma cells from non-diseased plasma cells. Exemplary multiparameteric flow cytometry techniques are described in Kumar S et al. 2016. In some embodiments, the multiparametric flow cytometry comprises gating for any one or more of CD138, CD38, CD45, CD56, CD19, cytoplasmic κ and λ immunoglobulin light chains, CD20, CD27, CD28, CD81, CD117, CD200, CD54, CD229, CD319, and VS38c. In particular embodiments, the multiparametric flow cytometry comprises gating for any one or more of CD138, CD38, CD45, CD56, CD19, and cytoplasmic κ and λ immunoglobulin light chains. In other particular embodiments, the multiparametric flow cytometry comprises gating for CD19, CD45, CD56, CD81, CD27, CD117, and cytoplasmic κ and λ immunoglobulin light chains. In preferred embodiments, the multiparametric flow cytometry comprises gating for CD138. In some embodiments, wherein the subject had been previously treated with an anticancer therapy comprising a monoclonal antibody against CD38 or CD138, the multiparametric flow cytometry comprises gating for any one or more of CD54, CD229, CD319, and VS38c. Exemplary multiparametric flow cytometry techniques are described in Kumar S et al. 2016, Flores-Montero J et al. 2017, Mishima Y et al. 2017, US20180140664A1, and WO2017198879A1.

In some embodiments, determination of remission comprises use of ASO-qPCR to detect presence or absence of multiple myeloma cells in the bone marrow sample. Exemplary ASO-qPCR techniques are described in Kumar S et al. 2016.

In some embodiments, determination of remission comprises use of next generation sequencing to detect presence or absence of multiple myeloma cells in the bone marrow sample. The next generation sequencing may comprise sequencing of immunoglobulin gene segments. Exemplary next generation sequencing techniques are described in Kumar S et al. 2016.

Additional conventional factors which may be considered when determining whether a subject is in complete remission from multiple myeloma includes assessment of hypercalcaemia, assessment of renal insufficiency, and assessment of anemia. Such criteria are described in Rajkumar S et al. 2014.

Methods for Disease Prognosis

The methods described herein are also useful for disease prognosis in a subject in need thereof. Prognosis can include predicting the outcome of the subject's disease, chance of recovery from the disease, response to a course of therapy, or tracking the progression of the disease. Prognosis can also include determining a course of therapy for the subject, based, e.g., on genetic profile of a subject's diseased cell, disease-associated exosomes, disease-associated transcriptomes, or diseased polynucleotide.

Accordingly, in some aspects provided herein is a method of prognosing a subject in need thereof, comprising genetically profiling one or more diseased cells, disease-associated exosomes, disease-associated transcriptomes, or diseased circulating polynucleotides that have been obtained from a fluid sample obtained from the subject, the subject having been previously administered a mobilizing agent disclosed herein in an amount effective to stimulate release of the one or more diseased cells, disease-associated exosomes, disease-associated transcriptomes, or diseased circulating polynucleotides into circulation, wherein the genetic profile is used in prognosis of the subject. In some embodiments, the method comprises administration of the mobilizing agent to the subject, collection of the fluid sample from the subject, and genetically profiling diseased cells, disease-associated exosomes, disease-associated transcriptomes, or diseased polynucleotides released into the fluid sample.

In some embodiments, the method comprises collection of a first fluid sample from the subject and profiling of one or more diseased cells, disease-associated exosomes, disease-associated transcriptomes, administration of an intervening agent (e.g., an mobilizing agent) to the subject, and collection of a second fluid sample from the subject and profiling of one or more diseased cells, disease-associated exosomes, disease-associated transcriptomes.

In some embodiments of any of the prognostic methods disclosed herein, the subject is determined to have no disease. In some embodiments of any of the prognostic methods disclosed herein, the subject is determined to have the disease. In some embodiments, the subject is determined to have cancer. In some embodiments, the subject is determined to not be in remission, or is suspected of harboring active disease.

In some embodiments, a report of the determination of a subject's prognosis according to any of the methods disclosed herein is transmitted over a network.

Methods for Disease Detection

The methods described herein are also useful for disease detection in a subject in need thereof. For example, the methods described herein are useful for detecting disease in the subject at very early stages of the disease, prior to when the disease would be detectable by otherwise conventional means.

Accordingly, provided herein is a method of detecting a disease in a subject, comprising detecting one or more diseased cells, disease-associated exosomes, disease-associated transcriptomes, or disease-associated polynucleotides in a fluid sample obtained from a subject, wherein the subject was previously administered an intervening agent in an amount effective to stimulate release of the one or more diseased cells, disease-associated exosomes, disease-associated transcriptomes, or disease-associated polynucleotides into circulation, and wherein the detection of the one or more diseased cells, disease-associated exosomes, disease-associated transcriptomes, or disease-associated polynucleotides is indicative of the disease in the subject.

In some embodiments, the method comprises collection of a first fluid sample from the subject and detection of one or more diseased cells, disease-associated exosomes, disease-associated transcriptomes, administration of an intervening agent to the subject, and collection of a second fluid sample from the subject and detection of one or more diseased cells, disease-associated exosomes, disease-associated transcriptomes. In some embodiments, the detection of the one or more diseased cells, disease-associated exosomes, disease-associated transcriptomes, or disease-associated polynucleotides in the second fluid sample is indicative of the disease in the subject. In some embodiments, the nondetection of diseased cells, disease-associated exosomes, disease-associated transcriptomes, or disease-associated polynucleotides in the second fluid sample is indicative of the absence of disease in the subject.

In some embodiments, the disease is a relapse of the disease. In some embodiments, the disease is cancer.

In some embodiments, the diseased cells comprise tumor cells. In some embodiments, the disease-associated exosomes are tumor exosomes. In some embodiments, the disease-associated transcriptomes are tumor transcriptomes. In some embodiments, the disease-associated polynucleotides are tumor-associated polynucleotides, e.g., tumor DNA.

In some embodiments, the intervening agent is a mobilizing agent. In some embodiments, the mobilizing agent comprises one or more cytokines or growth factors. In some embodiments, the second sample is a fluid sample. In some embodiments, the interval between collection of the first and the second samples is 1-30 days.

A skilled artisan, e.g., a clinician, may determine the timepoints for obtaining the samples and administering the intervening agent to the subject according to any conventional criteria known in the art.

Diseased cells (e.g., tumor cells or bacterial cells), disease-associated exosomes (e.g., tumor exosomes or bacterial exosomes), disease-associated transcriptomes (e.g., tumor transcriptomes or bacterial transcriptomes), and/or disease-associated polynucleotides (e.g., tumor DNA or bacterial DNA) may be analyzed using any methods known in the art, such as flow cytometry, PCR (e.g., qPCR or ASO-qPCR), sequencing technologies (e.g., next-generation sequencing, single-cell sequencing), immunostaining, immunohistochemistry, or immunofluorescence.

Methods for Actionable Information

The methods described herein can be used for obtaining actionable information in a subject. Such methods are particularly advantageous because mobilization of diseased cells (e.g., tumor cells), disease-associated exosomes (e.g., tumor exosomes), disease-associated transcriptomes (e.g., tumor transcriptomes), and/or disease-associated polynucleotides (e.g., tumor DNA) enhances yield of the diseased cells, disease-associated exosomes, disease-associated transcriptomes, and/or disease-associated polynucleotides obtained from a fluid sample, thereby enhancing sensitivity of the assay. In a patient with widespread (metastatic) disease, cLB may not disclose heterogeneity in different sites—whereas LBYE+ will—providing additional actionable information.

Accordingly, provided herein is a method of obtaining actionable information in a subject, comprising detecting one or more cancer biomarkers in a subject in need thereof, comprising analyzing diseased cells (e.g., tumor cells or bacterial cells), disease-associated exosomes (e.g., tumor exosomes or bacterial exosomes), disease-associated transcriptomes (e.g., tumor transcriptomes or bacterial transcriptomes), and/or disease-associated polynucleotides (e.g., tumor DNA or bacterial DNA) from one or more fluid or tissue samples obtained from the subject and thereby determining presence or absence of or change in the one or more markers (e.g., cancer biomarkers), wherein the subject is administered an intervening agent to stimulate release of the one or more diseased cells, disease-associated polynucleotides, or disease-associated polynucleotides into circulation. In some embodiments, tumor cells, tumor exosomes, tumor transcriptomes, or tumor DNA from at least two samples obtained from the subject are analyzed. In some embodiments, at least one fluid samples is obtained and diseased cells (e.g., tumor cells or bacterial cells), disease-associated exosomes (e.g., tumor exosomes or bacterial exosomes), disease-associated transcriptomes (e.g., tumor transcriptomes or bacterial transcriptomes), and/or disease-associated polynucleotides (e.g., tumor DNA or bacterial DNA) analyzed after the subject is administered the intervening agent. Accordingly, in some embodiments, the subject is administered one or more cytokines or growths factor prior to obtaining a fluid sample from the subject, e.g., 1-96 hours prior to obtaining a fluid sample from the subject. In some embodiments, a fluid or tissue sample is obtained and diseased cells (e.g., tumor cells or bacterial cells), disease-associated exosomes (e.g., tumor exosomes or bacterial exosomes), disease-associated transcriptomes (e.g., tumor transcriptomes or bacterial transcriptomes), and/or disease-associated polynucleotides (e.g., tumor DNA or bacterial DNA) analyzed prior to the subject administered one or more cytokines or growth factors. According, in some embodiments, the subject is administered the cytokine or growth factor after obtaining a fluid or tissue sample from the subject, e.g., 1-30 days after obtaining a fluid sample from the subject.

In some embodiments, the method comprises collection of a first sample from the subject, administration of an intervening agent to the subject, collection of a second sample from the subject, and analyzing change in a biomarker. In some embodiments, the intervening agent is a mobilizing agent. In some embodiments, the mobilizing agent comprises one or more cytokines or growth factors. In some embodiments, the second sample is a fluid sample. In some embodiments, the interval between collection of the first and the second samples is 1-30 days.

A skilled artisan, e.g., a clinician, may determine the timepoints for obtaining the samples and administering the intervening agent to the subject according to any conventional criteria known in the art.

Diseased cells (e.g., tumor cells or bacterial cells), disease-associated exosomes (e.g., tumor exosomes or bacterial exosomes), disease-associated transcriptomes (e.g., tumor transcriptomes or bacterial transcriptomes), and/or disease-associated polynucleotides (e.g., tumor DNA or bacterial DNA) may be analyzed using any methods known in the art, such as flow cytometry, PCR (e.g., qPCR or ASO-qPCR), sequencing technologies (e.g., next-generation sequencing, single-cell sequencing), immunostaining, immunohistochemistry, or immunofluorescence.

In some embodiments, the biomarkers may be hormones, proteins, genes, gene mutations, genome-wide fragmentation profiles, genetic amplifications or translocations, variant allele frequency (VAF), tumor mutational burden (TMB), microsatellite instability (MSI), DNA methylation pattern, and/or dysbiosis.

Methods for Predicting Treatment Response and Aiding Treatment Decision

The methods described herein can be used for predicting treatment response and/or aiding treatment decision, particularly following determining presence or absence of or change in one or more cancer biomarkers according to methods described herein,

Accordingly, in some aspects, provided herein is a method of predicting treatment response and/or aiding treatment decision for a subject in need thereof, comprising determining presence or absence of or change in one or more biomarkers (e.g., cancer biomarkers) in one or more fluid or tissue samples obtained from the subject, the subject having been administered a mobilizing agent disclosed herein in an amount effective to stimulate release of the one or more diseased cells (e.g., tumor cells or bacterial cells), disease-associated exosomes (e.g., tumor exosomes or bacterial exosomes), disease-associated transcriptomes (e.g., tumor transcriptomes or bacterial transcriptomes), and/or disease-associated polynucleotides (e.g., tumor DNA or bacterial DNA) into circulation, wherein the presence or absence of or change in one or more biomarkers is used in aiding treatment decision.

In some embodiments, the method comprises collection of a first sample from the subject, administration of the mobilizing agent to the subject, collection of a second sample from the subject, analyzing change in a biomarker, and predicting treatment response. In some embodiments, the method comprises collection of a first sample from the subject, administration of the mobilizing agent to the subject, collection of a second sample from the subject, analyzing change in a biomarker, and making a treatment decision. In some embodiments, the method directs treatment towards strategy/agents that the previous investigations including a cLB guided/taught away from.

In some embodiments, the subject is determined to have a disease or disease recurrence. In some embodiments, the subject is determined to have a cancer or cancer recurrence. In some embodiments, the subject is determined to not be in remission, or is suspected of harboring active disease. In some embodiments, the subject is determined to in serologic CR but not LBYE CR.

A skilled artisan, e.g., a clinician, may predict treatment response (e.g., to a cell-based or antibody-based therapy) and/or make treatment decision (e.g., prescribing a cell-based or antibody-based therapy) according to any conventional criteria with respect to biomarkers known in the art. For example, in some embodiments, for a subject with high TMB, the skilled artisan may predict the subject to likely respond to an immunotherapy in cancer and/or decide to prescribe an immunotherapy to the subject. As a further example, in some embodiments, the skilled artisan may decide to administer an agent such that the immune system of the subject is activated to target the identified biomarker.

In some embodiments, a report of the determination of a subject's prognosis according to any of the methods disclosed herein is transmitted over a network.

Methods of Treatment

In some aspects, provided herein are methods of treatment. The method of treatment may comprise a method of treating cancer. In some embodiments, the method of treating cancer comprises administering at least one cancer therapeutic to the subject if one or more tumor cells, tumor exosomes, tumor transcriptomes, or tumor DNA has been detected in a fluid sample obtained from the subject following administration of a mobilizing agent disclosed herein in an amount effective to stimulate release of the one or more tumor cells, tumor exosomes, tumor transcriptomes, or tumor DNA into circulation of the subject. In some embodiments, the method of treating cancer comprises administering at least one cancer therapeutic to the subject if one or more cancer biomarkers or change in one or more cancer biomarkers in a sample obtained from the subject following administration of an intervening agent in an amount effective to stimulate release of the one or more tumor cells, tumor exosomes, tumor transcriptomes, or tumor DNA into circulation of the subject or to induce an unexpected/unknown change in the biological characteristics exhibited. Exemplary anticancer therapeutics (cancer therapeutics) are disclosed herein. In some embodiments, the cancer therapeutic may be an immunotherapeutic agent (e.g., anticancer T cell, CAR-T cell, cancer vaccine, immunomodulator, checkpoint inhibitor, oncolytic virus, or targeted antibody), a chemotherapeutic agent, a radiotherapeutic agent, a hormone, a stem cell, or any combination thereof. In some embodiments, the anticancer therapeutic comprises an HSC transplant. In some embodiments, the anticancer therapeutic comprises an autologous HSC transplant. In particular embodiments, the anticancer therapeutic does not comprise an autologous HSC transplantation. In some embodiments, the anticancer therapeutic comprises an allogeneic HSC transplant. In some embodiments, the anticancer intervention comprises immunotherapy (including but not limited to cell therapy such as CAR-T cells, immune activating agents such as checkpoint inhibitors, and various combinations thereof).

In some embodiments, methods of treatment comprise determining a biomarker of one or more diseased cells, disease-associated exosomes, disease-associated transcriptomes, or disease-associated polynucleotides obtained from a subject via LBYE, and administering an anticancer therapeutic to the subject based on the determined biomarker. In some embodiments, biomarker may be a cancer biomarker, e.g., a hormone, a protein, a gene, a gene mutation, a genetic amplification or translocation, a mutational or genetic profile, a genome-wide fragmentation profile, variant allele frequency (VAF), tumor mutational burden (TMB), microsatellite instability (MSI), DNA methylation pattern, or dysbiosis. In some embodiments, methods of treatment comprise determining a mutational or genetic profile of one or more diseased cells, disease-associated exosomes, disease-associated transcriptomes, or disease-associated polynucleotides obtained from a subject via LBYE, and administering an anticancer therapeutic to the subject based on the determined genetic profile.

Pharmaceutical Compositions

Methods for treatment are also encompassed by the present invention. Said methods of the invention include administering a therapeutically effective amount of a therapeutic, e.g., an anticancer drug to the subject. The therapeutic can be formulated in pharmaceutical compositions. These compositions can comprise, in addition to one or more active ingredients, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, intramuscular or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, Lactated Ringer's injection. Preservatives, stabilizers, buffers, antioxidants and/or other additives can be included, as required.

Whether it is a polypeptide, antibody, nucleic acid, small molecule or other pharmaceutically useful compound that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of the disease being treated. Prescription of treatment, e.g. decisions on dosage etc., is within the responsibility of the cancer specialist (hematologist or oncologist) or other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are described in, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: LBYE and LBYE+ for Multiple Myeloma Patients

### Example 1A: LBYE for MRD Detection

Human patients who have undergone an anticancer treatment for multiple myeloma are split into two experimental groups, an “LBYE” and “control group”. Patients may be matched across experimental groups by age, sex, and other clinical factors, such as multiple myeloma burden or genetic profile of the multiple myeloma in the patient.

When in complete remission using conventional techniques after appropriate therapy, patients in the control group undergo a blood as well as bone marrow examination each of which is subjected to MRD detection/assay using the ClonoSEQ® technique (Adaptive Biotechnologies). A comparable “LBYE” experimental group is administered a single dose of motixafortide (0.5-2 mg/kg), a single dose of MGTA-145 (0.0075-0.3 mg/kg), or a single dose of MGTA-145 (0.0075-0.3 mg/kg) in combination with a single dose of plerixafor (0.24 mg/kg). 1-72 hours after administration, patients in the LBYE group undergo a blood draw. The blood samples are subjected to MRD assay using the ClonoSEQ® technique. Residual disease is detected in a greater number of patients in the LBYE group than in the control group, indicating successful detection/mobilization of hidden diseased cells in the patients undergoing LBYE sampling.

In another variation of the experiment, where myeloma patients serve as their own controls, patients undergo bone marrow examination after attaining complete remission. The marrow is subjected to MRD assay by ClonoSEQ®. After the marrow exam is done, each patient receives a single dose of motixafortide (0.5-2 mg/kg), a single dose of MGTA-145 (0.0075-0.3 mg/kg), or a single dose of MGTA-145 (0.0075-0.3 mg/kg) in combination with a single dose of plerixafor (0.24 mg/kg), and the blood is sampled 1-72 hours later. Blood samples are subjected to MRD assay by ClonoSEQ®. A greater proportion of patients is found to be positive for MRD from the blood after the LBYE technique than the bone marrow. All or most patients positive for MRD from the bone marrow are positive for MRD from the blood after LBYE technique, whereas a number of patients who are negative for MRD from the bone marrow are positive from the blood after the LBYE technique.

Cell-free DNA (cfDNA) is isolated from patient plasma samples according to known methods.

The concentration of the isolated cfDNA from LBYE and control patients are determined. Patients in the LBYE group exhibit higher cfDNA concentrations than patients in the control group, indicating successful mobilization of diseased ctDNA in the patients undergoing LBYE sampling, and indicating improved sensitivity for detecting MRD utilizing the LBYE methods disclosed herein and indicating improved sensitivity for detecting MRD utilizing the LBYE methods disclosed herein.

The cfDNA is subjected to whole exome sequencing or targeted sequencing to determine mutational profile of the cfDNA sample. Mutational profile of the cfDNA sample is, in some cases, compared to mutational profile of multiple myeloma cells obtained from bone marrow sample from the same patient.

### Example 1B: Patient Follow Up Following LBYE for MRD Detection

Human patients for which CTCs or ctDNA were detected by LBYE as described in Example 1A are administered a further course of anticancer treatment and monitored for relapse. The monitoring may comprise clinical assessment for relapse by any means known in the art. In some cases, the patients undergo further LBYE sampling and analysis, according to the methods described in Example 1A.

Human patients for which CTCs or ctDNA were not detected by LBYE as described in Example 1A are not administered a further course of anticancer treatment. Such patients are further monitored for presence or absence of CTCs and/or ctDNA at regular intervals using the methods described in Example 1A. The clinician determines the frequency and duration of the monitoring by the methods described in Example 1A, which can range from, e.g., once a month, once every two months, once every three months, once every four months, once every 6 months, once a year, for 1-10 years, 1-5 years, 1-3 years, or any of the subranges within 1-10 years.

### Example 1C: LBYE for Active Disease Prognosis

Human patients who are diagnosed with multiple myeloma are split into two experimental groups, an “LBYE” group and a “control group”. Patients may be matched across experimental groups by age, sex, and other clinical factors, such as multiple myeloma burden or genetic profile of the multiple myeloma in the patient.

Patients in the “LBYE” experimental group are administered a single dose of motixafortide (0.5-2 mg/kg), a single dose of MGTA-145 (0.0075-0.3 mg/kg), or a single dose of MGTA-145 (0.0075-0.3 mg/kg) in combination with a single dose of plerixafor (0.24 mg/kg), and undergo blood sampling 1-72 hours later. This sample is analyzed for the presence of various mutations and genetic variations known to influence prognosis in myeloma. Patients in the control group undergo a bone marrow examination that is subjected to similar testing. Patients in the LBYE group show a greater frequency and breadth of abnormalities than those in the control group. In a variation on the above experiment, the LBYE group patients undergo a bone marrow examination which is subjected to appropriate testing. The final result of the LBYE group is the sum total of findings from the blood and the marrow of each patient—and shows greater amount of relevant information than the control group subjected to bone marrow sampling alone.

In another variation on the experiment, the LBYE group's marrow and blood sample are pooled and analyzed in a single assay to make the test more cost-effective. Blood samples are separated a plasma fraction and cellular fractions. In yet another variation, only an LBYE group is studied and greater information found in the blood after LBYE compared to the bone marrow of the same patient. The cell fraction is analyzed for the presence of multiple myeloma CTCs by multiparametric flow cytometry according to methods described herein. Multiple myeloma CTCs are genetically profiled, e.g., by sequencing to determine the mutational profile of the CTCs. Mutational profile of the CTC sample is, in some cases, compared to mutational profile of multiple myeloma cells obtained from bone marrow sample from the same patient.

In some cases, cell-free DNA (cfDNA) is isolated from patient plasma samples according to known methods and genetically profiled, e.g., by sequencing. Mutational profile of the cfDNA sample is, in some cases, compared to mutational profile of multiple myeloma cells obtained from bone marrow sample from the same patient. It is conceivable that the LBYE approach consistently results in identification of at least all abnormalities seen in the bone marrow. If that is the case, LBYE will pioneer a “no marrow” approach—making bone marrow examination, arguably the most unpleasant aspect of investigating hematologic malignancies, redundant.

The mutational profile of the CTCs and/or cfDNA is used by a clinician for the patient's prognosis and treatment choice.

In some cases, the genetic profile of the CTCs and/or cfDNA is further monitored at regular intervals using the methods described in this Example. The clinician determines the frequency and duration of the monitoring by the methods described in this example, which can range from, e.g., once a month, once every two months, once every three months, once every four months, once every 6 months, once a year, for 1-10 years, 1-5 years, 1-3 years, or any of the subranges within 1-10 years.

TABLE 1
LBYE and LBYE+ for multiple myeloma patients

Myeloma - state

| by conventional | LBYE | | LBYE+ | |
|---|---|---|---|---|
| testing | Positive | Negative | Positive | Negative |
| Diagnosis | | | Greater | Lack of |
| | | | information | heterogeneity |
| | | | than available | |
| | | | on the marrow - | |
| | | | showing | |
| | | | heterogeneity of | |
| | | | bone lesions | |
| Complete | Not in CR | CR confirmed | Not in CR | CR confirmed |
| remission (CR) | | | | |
| Relapse | | | Greater | Lack of |
| | | | information | heterogeneity |
| | | | than available | |
| | | | on the marrow - | |
| | | | showing | |
| | | | heterogeneity of | |
| | | | bone lesions | |
| Solitary | There may be | True solitary | There may be | True solitary |
| plasmacytoma - | systemic | disease | systemic | disease |
| diagnosis | disease | | disease | |
| Solitary | Local or | Ongoing | Local or | Ongoing |
| plasmacytoma - | systemic relapse | remission | systemic relapse | remission |
| monitoring after | | | | |
| therapy | | | | |

### Example 2: LBYE for Lymphoma Patients

### Example 2A: LBYE for MRD Detection

Human patients who have undergone an anticancer treatment for lymphoma are split into two experimental groups, an “LBYE” and “control group”. Patients may be matched across experimental groups by age, sex, and other clinical factors, such as lymphoma burden or genetic profile of the lymphoma in the patient.

When in complete remission using conventional techniques after appropriate therapy, patients in the control group undergo a blood as well as bone marrow examination each of which is subjected to MRD detection/assay using any of the techniques described earlier (including but not limited to flow cytometry, sequencing, etc.). Patients in the “LBYE” group is administered a single dose of motixafortide (0.5-2 mg/kg), a single dose of MGTA-145 (0.0075-0.3 mg/kg), or a single dose of MGTA-145 (0.0075-0.3 mg/kg) in combination with a single dose of plerixafor (0.24 mg/kg). 1-72 hours after administration, patients in the LBYE group undergo a blood draw. The blood samples are subjected to MRD assay. Residual disease is detected in a greater number of patients in the LBYE group than in the control group, indicating successful detection/mobilization of hidden diseased cells in the patients undergoing LBYE sampling.

In another variation of the experiment, where lymphoma patients serve as their own controls, patients undergo blood sampling after attaining complete remission. The blood is subjected to MRD assay. After the blood test is done, each patient receives a single dose of motixafortide (0.5-2 mg/kg), a single dose of MGTA-145 (0.0075-0.3 mg/kg), or a single dose of MGTA-145 (0.0075-0.3 mg/kg) in combination with a single dose of plerixafor (0.24 mg/kg), and the blood is sampled 1-72 hours later. These blood samples are subjected to MRD assay by the same technique. A greater proportion of patients is found to be positive for MRD from the blood after the LBYE technique than the non-LBYE blood sample.

Cell-free DNA (cfDNA) is isolated from patient plasma samples according to known methods.

The concentration of the isolated cfDNA from LBYE and control patients are determined. In the second experiment, where patients serve as their own controls, even amongst patients exhibiting the presence of cfDNA (i.e., positive for MRD), the concentration/amount of cfDNA is higher after LBYE indicating successful mobilization of diseased ctDNA in the patients undergoing LBYE sampling, and indicating improved sensitivity for detecting MRD utilizing the LBYE methods disclosed herein and indicating improved sensitivity for detecting MRD utilizing the LBYE methods disclosed herein.

The cfDNA is subjected to whole exome sequencing or targeted sequencing to determine mutational profile of the cfDNA sample. Mutational profile of the cfDNA sample is, in some cases, compared to mutational profile of lymphoma cells obtained from bone marrow sample from the same patient.

### Example 2B: Patient Follow Up Following LBYE for MRD Detection

Human patients for which CTCs or ctDNA were detected by LBYE as described in Example 2A are administered a further course of anticancer treatment and monitored for relapse. The monitoring may comprise clinical assessment for relapse by any means known in the art. In some cases, the patients undergo further LBYE sampling and analysis, according to the methods described in Example 2A.

Human patients for which CTCs or ctDNA were not detected by LBYE as described in Example 2A are not administered a further course of anticancer treatment. Such patients are further monitored for presence or absence of CTCs and/or ctDNA at regular intervals using the methods described in Example 2A. The clinician determines the frequency and duration of the monitoring by the methods described in Example 2A, which can range from, e.g., once a month, once every two months, once every three months, once every four months, once every 6 months, once a year, for 1-10 years, 1-5 years, 1-3 years, or any of the subranges within 1-10 years.

### Example 2C: LBYE for Active Disease Prognosis

Human patients who are diagnosed with lymphoma are split into two experimental groups, an “LBYE” group and a “control group”. Patients may be matched across experimental groups by age, sex, and other clinical factors, such as lymphoma burden or genetic profile of the lymphoma in the patient.

Patients in the “LBYE” experimental group are administered a single dose of motixafortide (0.5-2 mg/kg), a single dose of MGTA-145 (0.0075-0.3 mg/kg), or a single dose of MGTA-145 (0.0075-0.3 mg/kg) in combination with a single dose of plerixafor (0.24 mg/kg), and undergo blood sampling 1-72 hours later. This sample is analyzed for the presence of various mutations and genetic variations known to influence prognosis in myeloma. Patients in the control group undergo a bone marrow examination that is subjected to similar testing. Patients in the LBYE group show a greater frequency and breadth of abnormalities than those in the control group. In a variation on the above experiment, the LBYE group patients undergo a bone marrow examination which is subjected to appropriate testing. The final result of the LBYE group is the sum total of findings from the blood and the marrow of each patient—and shows greater amount of relevant information than the control group subjected to bone marrow sampling alone.

In another variation on the experiment, the LBYE group's marrow and blood sample are pooled and analyzed in a single assay to make the test more cost-effective. In yet another variation, only an LBYE group is studied and grater information found in the blood after LBYE compared to the bone marrow of the same patient. The mononuclear cell fraction is analyzed for the presence of lymphoma CTCs by multiparametric flow cytometry according to methods described herein. Lymphoma CTCs are genetically profiled, e.g., by sequencing to determine the mutational profile of the CTCs. Mutational profile of the CTC sample is, in some cases, compared to mutational profile of lymphoma cells obtained from bone marrow sample from the same patient.

In some cases, cell-free DNA (cfDNA) is isolated from patient plasma samples according to known methods and genetically profiled, e.g., by sequencing. Mutational profile of the cfDNA sample is, in some cases, compared to mutational profile of lymphoma cells obtained from bone marrow sample from the same patient.

The mutational profile of the CTCs and/or cfDNA is used by a clinician for the patient's prognosis and treatment choice.

In some cases, the genetic profile of the CTCs and/or cfDNA is further monitored at regular intervals using the methods described in this Example. The clinician determines the frequency and duration of the monitoring by the methods described in this example, which can range from, e.g., once a month, once every two months, once every three months, once every four months, once every 6 months, once a year, for 1-10 years, 1-5 years, 1-3 years, or any of the subranges within 1-10 years.

### Example 3: LBYE for Solid Tumor Patients

### Example 3A: LBYE for MRD Detection

Human patients who have undergone an anticancer treatment for a specific solid tumor (e.g., lung cancer, breast cancer, colon cancer, amongst others) are split into two experimental groups, an “LBYE” and “control group”. Patients may be matched across experimental groups by age, sex, and other clinical factors, such as solid tumor burden or genetic profile of the solid tumor in the patient.

When in complete remission using conventional techniques after appropriate therapy, patients in the control group undergo a blood examination each of which is subjected to MRD detection/assay using an appropriate assay (e.g., the Guardant 360 liquid biopsy or the Tempus XF liquid biopsy). Patients in the “LBYE” group is administered a single dose of MGTA-145 (0.0075-0.3 mg/kg) or a single dose of MGTA-145 (0.0075-0.3 mg/kg) in combination with a single dose of plerixafor (0.24 mg/kg). 1-72 hours after MGTA-145 administration, patients in the LBYE group undergo a blood draw. The blood samples are subjected to the same MRD assay. Residual disease is detected in a greater number of patients in the LBYE group than in the control group, indicating successful detection/mobilization of hidden diseased cells in the patients undergoing LBYE sampling.

In another variation of the experiment, where cancer patients serve as their own controls, patients undergo blood examination after attaining complete remission. Afterwards, each patient receives a single dose of MGTA-145 (0.0075-0.3 mg/kg) or a single dose of MGTA-145 (0.0075-0.3 mg/kg) in combination with a single dose of plerixafor (0.24 mg/kg), and the blood is sampled 1-72 hours later. Both samples are subjected to MRD assay. A greater proportion of patients is found to be positive for MRD from the blood sample obtained after LBYE treatment, as compared to the non-LBYE blood sample.

Cell-free DNA (cfDNA) is isolated from patient plasma samples according to known methods.

The concentration of the isolated cfDNA from LBYE and control patients are determined. Patients in the LBYE group exhibit higher cfDNA concentrations than patients in the control group, indicating successful mobilization of diseased ctDNA in the patients undergoing LBYE sampling, and indicating improved sensitivity for detecting MRD utilizing the LBYE methods disclosed herein and indicating improved sensitivity for detecting MRD utilizing the LBYE methods disclosed herein.

The cfDNA is subjected to whole exome sequencing or targeted sequencing to determine mutational profile of the cfDNA sample. Mutational profile of the cfDNA sample is, in some cases, compared to mutational profile of solid tumor cells obtained from the solid tumor sample from the same patient.

### Example 3B: Patient Follow Up Following LBYE for MRD Detection

Human patients for which CTCs or ctDNA were detected by LBYE as described in Example 3A are administered a further course of anticancer treatment and monitored for response and relapse. The monitoring may comprise clinical assessment for response and relapse by any means known in the art. In some cases, the patients undergo further LBYE sampling and analysis to decide about further treatment, according to the methods described in Example 3A.

Human patients for which CTCs or ctDNA were not detected by LBYE (“LBYE CR”) as described in Example 3A are not administered a further course of anticancer treatment. Such patients are further monitored for presence or absence of CTCs and/or ctDNA at regular intervals using the methods described in Example 3A. The clinician determines the frequency and duration of the monitoring by the methods described in Example 3A, which can range from, e.g., once a month, once every two months, once every three months, once every four months, once every 6 months, once a year, for 1-10 years, 1-5 years, 1-3 years, or any of the subranges within 1-10 years.

### Example 3C: LBYE for Active Disease Prognosis

Human patients who are diagnosed with one or more solid tumors are split into two experimental groups, an “LBYE” group and a “control group”. Patients may be matched across experimental groups by age, sex, and other clinical factors, such as solid tumor burden or genetic profile of the solid tumor in the patient.

Patients in the “LBYE” experimental group are administered a single dose of MGTA-145 (0.0075-0.3 mg/kg) or a single dose of MGTA-145 (0.0075-0.3 mg/kg) in combination with a single dose of plerixafor (0.24 mg/kg), and undergo blood sampling 1-72 hours later. This sample is analyzed for the presence of various mutations and genetic variations known to influence prognosis. Patients in the control group undergo a similar examination of the solid tumor tissue or a conventional liquid biopsy that is subjected to similar testing. Patients in the LBYE group show a greater frequency and breadth of abnormalities than those in the control group. In a variation on the above experiment, the LBYE group patients undergo a solid tumor examination which is subjected to appropriate testing. The final result of the LBYE group is the sum total of findings from the blood and the solid tumor tissue of each patient—and shows greater amount of relevant information than the control group subjected to solid tumor sampling alone.

In another variation on the experiment, the LBYE group's tumor and blood samples are pooled and analyzed in a single assay to make the test more cost-effective. In yet another variation, only an LBYE group is studied and greater information found in the blood after LBYE compared to the solid tumor of the same patient. The mononuclear cell fraction is analyzed for the presence of solid tumor CTCs by multiparametric flow cytometry according to methods described herein. Solid tumor CTCs are genetically profiled, e.g., by sequencing to determine the mutational profile of the CTCs. Mutational profile of the CTC sample is, in some cases, compared to mutational profile of solid tumor cells obtained from solid tumor sample from the same patient.

In some cases, cell-free DNA (cfDNA) is isolated from patient plasma samples according to known methods and genetically profiled, e.g., by sequencing. Mutational profile of the cfDNA sample is, in some cases, compared to mutational profile of solid tumor cells obtained from solid tumor sample from the same patient.

The mutational profile of the CTCs and/or cfDNA is used by a clinician for the patient's prognosis and treatment choice.

In some cases, the genetic profile of the CTCs and/or cfDNA is further monitored at regular intervals using the methods described in this Example. The clinician determines the frequency and duration of the monitoring by the methods described in this example, which can range from, e.g., once a month, once every two months, once every three months, once every four months, once every 6 months, once a year, for 1-10 years, 1-5 years, 1-3 years, or any of the subranges within 1-10 years.

### Example 4: LBYE for Screening Healthy Individuals (Including Survivors of Prior Cancer Who are in Remission—and are at Risk of a New Malignancy at a Greater Frequency than Expected in Healthy Individuals)

Individuals who are not known to have cancer undergo liquid biopsy as a screening tool. An unsuspected malignancy may be detected in a small proportion of such subjects. The same set of subjects undergoes screening after the LBYE technique. An unsuspected/undiagnosed malignancy is found in a greater proportion of subjects.

While the invention has been particularly shown and described with reference to a preferred embodiment and various alternate embodiments, it will be understood by persons skilled in the relevant art that various changes in form and details can be made therein without departing from the spirit and scope of the invention.

### Example 5: LBYE+ for Biomarker Detection and Aiding Treatment Decision

Human subjects who are enrolled and may be matched across experimental groups by age, sex, and other clinical factors, such as tumor burden or genetic profile of the tumor in the patient.

A sample from the subjects is collected at the time of diagnosis (“baseline sample”) and analyzed for tumor mutational burden (TMB) and microsatellite instability (MSI). Subjects are then administered a single dose of plerixafor (10-20 mg), a single dose of motixafortide (0.5-2 mg/kg), a single dose of MGTA-145 (0.0075-0.3 mg/kg), or a single dose of MGTA-145 (0.0075-0.3 mg/kg) in combination with a single dose of plerixafor (0.24 mg/kg), and blood sample collected 1-72 hours later. This blood sample (“LBYE+ sample”) is analyzed for TMB and MSI. TMB and/or MSI of the LBYE+ sample is higher than that of the baseline sample, and suggests suitability for immunotherapy.

In another variation of the experiment, the baseline sample is collected from patients after the diagnosis and receiving treatment for a period of time, e.g., after one or more treatment cycles.

In variations of the experiment, other intervening agent than plerixafor is administered to the subject. Suitable intervening agents include, but are not limited to, mobilizing agents, chemotherapeutic agents, monoclonal antibodies, anticancer agents, nutrients (e.g., folate, vitamin B12, or vitamin D), or any agents that stimulate release of diseased cells, disease-associated exosomes, disease-associated transcriptomes, or diseased polynucleotides into circulation.

In variations of the experiment, other biomarkers than TMB of the baseline sample and the LBYE+ sample are analyzed. Suitable biomarkers for analysis include, but are not limited to, hormones, proteins, genes, gene mutations, genetic amplification or translocations, mutational or genetic profiles, variant allele frequency (VAF), microsatellite instability (MSI), and DNA methylation pattern.

In a particular variation of the experiment, a chemotherapeutic agent is administered to the subject as intervening agent, and VAF of the baseline sample and the LBYE+ sample is analyzed as biomarker. LBYE+ sample has a reduced VAF than the baseline sample, suggesting the chemotherapy is worth administering repeatedly for an appropriate length.

The change/difference of biomarker detected in the LBYE+ sample and the baseline sample is used by a clinician to predict treatment response and/or make treatment decision.

TABLE 2
LBYE+ for aiding treatment decision

| | | | |
|---|---|---|---|
| Subject | Baseline Sample | LBYE+ Sample | |
| Groups | Biomarker Status | Biomarker Change | Treatment Decision |

Healthy Subjects

| | | | |
|---|---|---|---|
| Low | Positive | No change in biological | Monitor |
| disase risk | | findings | |
| | Positive | Change in biological | Investigate further |
| | | findings | |
| | Negative | Not indicated | NA |
| High | Positive | No change in biological | Monitor or investigate |
| disease | | findings | further |
| risk | Positive | Change in biological | Investigate further |
| | | findings | |
| | Negative | No change in biological | Monitor |
| | | findings | |
| | Negative | Change in biological | Monitor or investigate |
| | | findings | further |

Subjects Diagosed with Tumor or Tumor Recurrence

| | | | |
|---|---|---|---|
| Active | No actionable data | No change in biological | No change in therapy |
| disease | | findings | |
| | No actionable data | Change in biological | Possible change in |
| | | findings | therapy |
| | Actionable data | No change in biological | No change in therapy |
| | | findings | |
| | Actionable data | Change in biological | Possible change in |
| | | findings | therapy |
| Remission | Positive | No change in biological | No change in therapy |
| | | findings | |
| | Positive | Change in biological | Possible change in |
| | | findings | therapy |
| | Negative | No change in biological | No change in therapy |
| | | findings | |
| | Negative | Change in biological | Monitor or investigate |
| | | findings | further |

TABLE 3
LBYE+ for predicting treatment response and/or treatment decision

| Patient | | | | | | | |
|---|---|---|---|---|---|---|---|
| Group/ | | Baseline | Baseline | | LBYE+ | | |
| Disease | | Sample | Sample | Intervening | Sample | LBYE Sample | Treatment |
| Phase | Biomarker | Status | Interpretation | Agent | Status | Interpretation | Decision |
| T cell | Genomic | TET2 | | Plerixafor | TET2 | | |
| lymphoma/ | variants | | | | | | |
| Partial | VAF | 23.80% | | | 20.20% | | |
| remission | MSI | Stable | | | Stable | | |
| | TMB | 0.4 | Immunotherapy | | 0.5 | Immunotherapy | No change |
| | | m/MB | unsuitable | | m/MB | unsuitable | |
| Colon | Genomic | APC | | Plerixafor | APC | | |
| Cancer/ | variants | | | | | | |
| Relapse | VAF | 7.80% | | | 7.30% | | |
| | Genomic | TP53 | | | TP53 | | |
| | variants | | | | | | |
| | VAF | 6.30% | | | 6.30% | | |
| | MSI | Stable | | | Stable | | |
| | TMB | 0.5 | Immunotherapy | | 1.1 | Immunotherapy | Change |
| | | m/MB | unsuitable | | m/MB | may be suitable | treatment |
| Gall | Genomic | KRAS | | G-CSF | | | |
| bladder | variants | | | | | | |
| Cancer/ | VAF | 15.20% | | | | | |
| Relapse | Genomic | | | | | | |
| | variants | | | | | | |
| | VAF | | | | | | |
| | MSI | Not | | | | | |
| | | assessible | | | | | |
| | TMB | 3.3 | Immunotherapy | | | Immunotherapy | No change |
| | | m/MB | may be suitable | | | suitable | |

1. A method of detecting one or more diseased cells, disease-associated exosomes, disease-associated transcriptomes, or disease-associated polynucleotides in a fluid sample obtained from a subject, comprising:
a. administering to the subject an effective amount of one or more cytokines, chemokines, or growth factors or coagulation system modulators;
b. obtaining the fluid sample from the subject; and
c. determining the presence or absence of one or more diseased cells, disease-associated exosomes, disease-associated transcriptomes, or disease-associated polynucleotides in the fluid sample,
wherein, if the detection is the detection of diseased cells or disease-associated polynucleotides, the cytokine is not plerixafor.

2. A method of identifying a disease in a subject thereof, comprising:
a. administering to the subject an effective amount of one or more cytokines, chemokines, or growth factors or coagulation system modulators;
b. obtaining a fluid sample from the subject; and
c. detecting the presence or absence of one or more diseased cells, disease-associated exosomes, disease-associated transcriptomes, or disease-associated polynucleotides in the fluid sample,
wherein the detection of the presence of one or more diseased cells, disease-associated exosomes, disease-associated transcriptomes, or disease-associated polynucleotides is indicative of the disease in the subject, and
wherein, if the detection is the detection of diseased cells or disease-associated polynucleotides, the cytokine is not plerixafor.

3. A method of prognosing a subject in need thereof, comprising:
a. administering to the subject an effective amount of one or more cytokines, chemokines, or growth factors or coagulation system modulators;
b. obtaining a fluid sample from the subject; and
c. detecting one or more diseased cells, disease-associated exosomes, disease-associated transcriptomes, or disease-associated polynucleotides in the fluid sample,
wherein the detection of diseased cells, disease-associated exosomes, disease-associated transcriptomes, or disease-associated polynucleotides is indicative of the subject's prognosis, wherein, if the detection is the detection of diseased cells or disease-associated polynucleotides, the cytokine is not plerixafor.

4. The method of any one of claims 1-3, wherein the one or more cytokines, chemokines, or growth factors comprises a CXCR4 antagonist, a growth-related gene product β (GROβ) or a fragment or analog thereof, or a combination thereof.

5. The method of any one of claims 1-3, wherein the one or more coagulation system modulators comprises heparin or a derivative thereof, a direct oral anticoagulant (DOAC), a tissue plasminogen activator (tPA), a streptokinase, an urokinase, or a plasminogen activator inhibitor-1 (PAI-1) modulator.

6. The method of any one of claims 1-5, wherein the steps are performed in the order of step a to step c.

7. A method of predicting disease recurrence in a subject, comprising the steps of:
a. obtaining a first fluid sample from the subject and analyzing the quantity of diseased cells, disease-associated exosomes, disease-associated transcriptomes, or disease-associated polynucleotides;
b. administering to the subject an effective amount an intervening agent;
c. obtaining a second fluid sample from the subject and analyzing the quantity of diseased cells, disease-associated exosomes, disease-associated transcriptomes, or disease-associated polynucleotide; and
d. determining change of quantity in the diseased cells, disease-associated exosomes, disease-associated transcriptomes, or disease-associated polynucleotides in the first and the second fluid samples; thereby predicting disease recurrence by evaluating the change.

8. The method of claim 7, wherein the subject is administered an effective amount of an intervening agent before the first sample is obtained.

9. The method of any one claims 1-8, wherein the fluid sample is a blood sample.

10. The method of claim 9, wherein the blood sample is a plasma or serum sample.

11. The method of claim 9 or 10, wherein the blood sample is a whole blood sample or a cellular fraction of a whole blood sample.

12. The method of any one claims 1-11, wherein the fluid sample is an ascites, cerebrospinal fluid, lymph, sweat, urine, tears, saliva, pleural fluid, pericardial fluid, bronchoalveolar fluid, cavity rinse or swab, or organ rinse or swab sample.

13. The method of any one claims 1-12, wherein the presence or absence or quantity of one or more diseased cells, disease-associated exosomes, disease-associated transcriptomes, or disease-associated polynucleotides in the fluid sample is determined using one or more methods selected from: flow cytometry, PCR (e.g., qPCR or ASO-qPCR), sequencing (e.g., next-generation sequencing, single-cell sequencing), immunostaining, immunohistochemistry, or immunofluorescence.

14. The method of any one claims 1-13, wherein the presence or absence or quantity of diseased cells, disease-associated exosomes, disease-associated transcriptomes, or disease-associated polynucleotides in the fluid sample is determined using a method with a sensitivity of at least 1 in 100,000 cells.

15. A method of obtaining actionable information in a subject, comprising the steps of:
a. obtaining a first sample from the subject and analyzing a biomarker;
b. administering to the subject an effective amount of an intervening agent;
c. obtaining a second sample from the subject and analyzing the biomarker; and
d. determining change of the biomarker in the first and the second samples, thereby obtaining actionable information.

16. The method of claim 15, wherein the analysis of the biomarker obtained from the first sample does not yield actionable information.

17. The method of claim 15 or 16, wherein the actionable information is different from information obtained from analyzing the biomarker obtained from the first sample.

18. A method of predicting treatment response of a subject, comprising the steps of:
a. obtaining a first sample from the subject and analyzing a biomarker;
b. administering to the subject an effective amount of an intervening agent;
c. obtaining a second sample from the subject and analyzing the biomarker; and
d. determining change of the biomarker in the first and the second samples, thereby predicting treatment response by evaluating the change of the biomarker.

19. The method of claim 18, wherein the prediction of treatment response is different from a prediction made from analyzing the biomarker obtained from the first sample.

20. A method of supporting treatment decision of a subject, comprising the steps of:
a. obtaining a first sample from the subject and analyzing a biomarker;
b. administering to the subject an effective amount an intervening agent;
c. obtaining a second sample from the subject and analyzing the biomarker; and
d. determining change of the biomarker in the first and the second samples; thereby making an optimal treatment decision by evaluating the change of the biomarker.

21. The method of claim 20, wherein the optimal treatment decision is different from a decision made from analyzing the biomarker obtained from the first sample.

22. The method of claim 20 or 21, wherein the treatment decision is starting a treatment, staying on current treatment, adjusting current treatment, stopping treatment, or switching to a different treatment.

23. The method of any one of claims 15-22, wherein the steps are performed in the order of step a to step d.

24. The method of any one of claims 15-23, wherein the biomarker is not detectable from the first sample.

25. The method of any one of claims 15-24, wherein the biomarker is a hormone, a protein, a gene, a gene mutation, a genetic amplification or translocation, a mutational or genetic profile, a genome-wide fragmentation profile, variant allele frequency (VAF), tumor mutational burden (TMB), microsatellite instability (MSI), DNA repair deficiency/defect, DNA methylation pattern, or dysbiosis.

26. The method of any one of claims 15-25, wherein analyzing the biomarker is determining presence or absence of the biomarker or determining qualitative and quantitative data of the biomarker.

27. The method of any one of claims 15-26, wherein analyzing the biomarker is determining presence or absence of a protein, a gene, a gene mutation, or dysbiosis, determining telomere length, thymidylate synthase expression, or hypomethylation, determining quantitative data of VAF or TMB, or determining qualitative data of MSI.

28. The method of any one of claims 15-27, wherein the biomarker is analyzed using one or more methods selected from: flow cytometry, PCR (e.g., qPCR or ASO-qPCR), sequencing (e.g., next-generation sequencing, single-cell sequencing), immunostaining, immunohistochemistry, or immunofluorescence.

29. The method of any one of claims 15-28, wherein the first sample obtained from the subject may is a tissue sample or a fluid sample.

30. The method of any one of claims 15-29, wherein the second sample is obtained from the subject is a fluid sample.

31. The method of claim 29 or 30, wherein the fluid sample is a blood sample.

32. The method of claim 31, wherein the blood sample is a plasma or serum sample.

33. The method of claim 31, wherein the blood sample is a whole blood sample or a cellular fraction of a whole blood sample.

34. The method of any one of claims 29-33, wherein the fluid sample is an ascites, cerebrospinal fluid, lymph, sweat, urine, tears, saliva, pleural fluid, pericardial fluid, bronchoalveolar fluid, cavity rinse or swab, or organ rinse or swab sample.

35. The method of any one of claims 15-34, wherein the second samples is obtained at least about 1 hour after the first sample is obtained.

36. The method of any one of claims 7-35, wherein the intervening agent is capable of stimulating release of one or more diseased cells, disease-associated exosomes, disease-associated transcriptomes, or disease-associated polynucleotides into circulation, optionally wherein the one or more diseased cells, disease-associated exosomes, disease-associated transcriptomes, or disease-associated polynucleotides are one or more tumor cells, tumor exosomes, tumor transcriptomes, or tumor DNA.

37. The method of any one of claims 7-36, wherein the intervening agent is a mobilizing agent, an anticancer agent, a chemotherapeutic agent, a monoclonal antibody, or a nutrient, or a combination thereof.

38. The method of any one of claims 7-37, wherein the intervening agent is one or more cytokines, chemokines, or growth factors or coagulation system modulators.

39. The method of claim 38, wherein the one or more cytokines, chemokines, or growth factors comprises erythropoietin or a variant or analog thereof, methoxy polyethylene glycol-epoetin beta, G-CSF, PEGylated G-CSF, GM-CSF, SCF, IL-3, KGF, plerixafor, a CXCR4 antagonist, a GROβ or a fragment or analog thereof, or a combination thereof.

40. The method of claim 38, wherein the one or more coagulation system modulators comprises wherein the one or more coagulation system modulators comprises heparin or a derivative thereof, a direct oral anticoagulant (DOAC), a tissue plasminogen activator (tPA), a streptokinase, an urokinase, a plasminogen activator inhibitor-1 (PAI-1) modulator, or a combination thereof.

41. The method of claim 38 or 39, wherein the intervening agent comprises plerixafor.

42. The method of claim 41, comprising administering 0.1-0.4 mg/kg plerixafor or about 10-25 mg plerixafor to the subject.

43. The method of claim 41 or 42, comprising administering about 0.16 mg/kg, about 0.24 mg/kg, about 10 mg plerixafor, about 13 mg plerixafor, or about 20 mg plerixafor to the subject.

44. The method of claim any one of claims 41-43, comprising administering plerixafor to the subject subcutaneously, intramuscularly, intravenously, or by inhalation.

45. The method of any one of claims 41-44, comprising administering plerixafor to the subject daily for 1-4 days.

46. The method of claim any one of claims 41-45, comprising administering plerixafor to the subject once prior to obtaining the second sample.

47. The method of claim 46, comprising administering plerixafor to the subject 4-96 hours prior to obtaining the second sample.

48. The method of claim 46 or 47, comprising administering plerixafor to the subject about 11 hours prior to obtaining the second sample.

49. The method of any one of claims 7-52, wherein the intervening agent comprises a cancer therapeutic.

50. The method of claim 38 or 39, wherein the intervening agent comprises a CXCR4 antagonist or GROβ or a fragment or analog thereof.

51. The method of any one of claim 1-14 or 50, wherein administering the CXCR4 antagonist or GROβ or a fragment or analog thereof to the subject stimulates release of one or more diseased cells, disease-associated exosomes, disease-associated transcriptomes, or disease-associated polynucleotide into circulation, optionally wherein the one or more diseased cells, disease-associated exosomes, disease-associated transcriptomes, or disease-associated polynucleotides is one or more tumor cells, tumor exosomes, tumor transcriptomes, or tumor DNA.

52. The method of any one of claim 1-14, 50 or 51, wherein the CRCX4 antagonist is motixafortide, balixafortide, or YF-H-2015005.

53. The method of any one of claim 1-14 or 50-52, wherein the GROβ or a fragment or analog thereof is MGTA-145.

54. The method of any one of claim 1-14 or 50-53, wherein the CXCR4 antagonist or GROβ or a fragment or analog thereof is co-administered with a cytokine, chemokine, or growth factor.

55. The method of claim 54, wherein co-administering the CXCR4 antagonist or GROβ or a fragment or analog thereof and the cytokine, chemokine, or growth factor to the subject stimulates release of one or more diseased cells, disease-associated exosomes, disease-associated transcriptomes, or diseased polynucleotide into circulation, optionally wherein the one or more diseased cells, disease-associated exosomes, disease-associated transcriptomes, or diseased polynucleotides is one or more tumor cells, tumor exosomes, tumor transcriptomes, or tumor DNA.

56. The method of claim 54 or 55, wherein the cytokine, chemokine, or growth factor is a CXCR4 antagonist.

57. The method of claim 54 or 55, wherein the cytokine, chemokine, or growth factor is selected from erythropoietin, G-CSF, GM-CSF, SCF, IL-3, KGF, motixafortide, balixafortide, YF-H-2015005, and plerixafor.

58. The method of claim 54 or 55, wherein the cytokine, chemokine, or growth factor is plerixafor.

59. The method of claim 54 or 55, wherein the cytokine, chemokine, or growth factor is G-CSF.

60. The method of any one of claim 1-14 or 50-59, wherein the GROβ or a fragment or analog thereof is MGTA-145, and wherein MGTA-145 is co-administered with a cytokine, chemokine, or growth factor, optionally wherein the cytokine, chemokine, or growth factor is plerixafor or G-CSF.

61. The method of any one of claim 1-14 or 50-60, wherein the GROβ or a fragment or analog thereof is MGTA-145, and wherein MGTA-145 is co-administered with plerixafor.

62. The method of any one of claim 1-14 or 50-61, wherein the subject has been administered or is administered 0.0075-0.3 mg/kg, 0.015-0.15 mg/kg, or 0.03-0.15 mg/kg MGTA-145.

63. The method of any one of claim 1-14 or 50-62, wherein the subject has been administered or is administered about 0.0075 mg/kg, about 0.015 mg/kg, about 0.03 mg/kg, about 0.075 mg/kg, about 0.15 mg/kg, or about 0.3 mg/kg MGTA-145.

64. The method of any one of claims 54-63, wherein the subject is co-administered 0.1-0.4 mg/kg plerixafor or 5-50 mg plerixafor.

65. The method of any one of claims 54-64, wherein the subject is co-administered about 0.16 mg/kg, about 0.24 mg/kg, about 13 mg, or about 20 mg plerixafor.

66. The method of any one of claims 54-64, wherein the subject is co-administered about 5 mg, about 10 mg, about 15 mg, or about 20 mg plerixafor.

67. The method of claim any one of claims 53-66, wherein MGTA-145 is administered intravenously.

68. The method of claim any one of claims 57-67, wherein plerixafor is administered subcutaneously, intramuscularly, intravenously, or by inhalation.

69. The method of any one of claim 1-14 or 50-60, wherein the CXCR4 antagonist is motixafortide, and wherein motixafortide is co-administered with G-CSF.

70. The method of any one of claims 1-14, 50-60 and 69, wherein the subject has been administered or is administered 0.5-2 mg/kg motixafortide.

71. The method of any one of claims 1-14, 50-60 and 69-70, wherein the subject has been administered or is administered about 0.5 mg/kg, about 0.75 mg/kg, about 1.0 mg/kg, about 1.25 mg/kg, about 1.5 mg/kg, or about 2 mg/kg motixafortide.

72. The method of any one of claims 54-60 and 69-71, wherein the subject is co-administered 5-20 μg/kg G-CSF.

73. The method of any one of claims 54-60 and 69-72, wherein the subject is co-administered about 10 μg/kg G-CSF.

74. The method of any one of claims 54-60 and 69-73, wherein the subject is co-administered about 100 μg, about 200 μg, about 250 μg, about 300 μg, about 480 μg, about 500 μg, about 1,000 μg G-CSF.

75. The method of any one of claims 54-60 and 69-74, wherein motixafortide is administered subcutaneously.

76. The method of any one of claims 54-58 and 69-75, wherein G-CSF is administered subcutaneously.

77. The method of any one of claims 1-76, wherein the subject has or is suspected to have a cancer or cancer recurrence.

78. The method of any one of claims 1-76, wherein the subject does not have a cancer or cancer recurrence.

79. The method of any one of claims 1-76, wherein the subject has no detectable cancer.

80. The method of any one of claims 1-76, wherein the subject has or is suspected to have a neurological condition.

81. The method of claim 80, wherein the neurological condition is Alzheimer's disease or Parkinson's disease.

82. The method of any one of claims 1-14 and 36-76, wherein the disease is a cancer or a neurological condition (e.g., Alzheimer's disease or Parkinson's disease).

83. The method of any one of claims 1-14 and 36-76, wherein the one or more diseased cells, disease-associated exosomes, disease-associated transcriptomes, or disease-associated polynucleotides are non-tumor-derived.

84. The method of any one of claim 83, wherein the one or more diseased cells, disease-associated exosomes, disease-associated transcriptomes, or disease-associated polynucleotides are one or more bacterial cells, bacterial exosomes, bacterial transcriptomes, or bacterial DNA.

85. The method of any one of claims 1-14 and 36-76, wherein the one or more diseased cells, disease-associated exosomes, disease-associated transcriptomes, or disease-associated polynucleotides are one or more tumor cells, tumor exosomes, tumor transcriptomes, or tumor DNA.

86. A method of detecting the presence or absence of minimal residual disease in a subject in need thereof, comprising determining presence or absent of one or more tumor cells, tumor exosomes, tumor transcriptomes, or tumor DNA, according to the method of claim 85, wherein:
a. presence of the one or more tumor cells, tumor exosomes, tumor transcriptomes, or tumor DNA in the fluid sample indicates presence of minimal residual disease in the subject, and
b. absence of the one or more tumor cells, tumor exosomes, tumor transcriptomes, or tumor DNA in the fluid sample indicates absence of minimal residual disease in the subject.

87. A method of treating cancer in a subject in need thereof, comprising
a. administering to the subject an effective amount of CXCR4 antagonist, GROβ or a fragment or analog thereof, or coagulation system modulator;
b. obtaining a fluid sample from the subject;
c. determining the presence or absence of one or more tumor cells, tumor exosomes, tumor transcriptomes, or tumor DNA in the fluid sample; and
d. administering at least one cancer therapeutic to the subject if presence of one or more tumor cells, tumor exosomes, tumor transcriptomes, or tumor DNA in the fluid sample is detected.

88. The method of claim 1 or 87, wherein the cancer therapeutic is not an autologous HSC transplant.

89. The method of any one of claims 77-88, wherein the cancer is primary cancer.

90. The method of any one of claims 77-89, wherein the cancer is solid tumor.

91. The method of any one of claims 77-90, wherein the cancer is selected from adrenal cancer, anal cancer, bile duct cancer, bladder cancer, bone cancer, cancer of the brain or central nervous system, basal cell skin cancer, breast cancer, cervical cancer, colorectal cancer, endometrial cancer, esophageal cancer, eye cancer, gallbladder cancer, gastrointestinal carcinoid tumors, gastrointestinal stromal tumor (GIST), gastric cancer, glioma, glioblastoma, head and neck cancer (including head and neck squamous cell carcinoma), Hodgkin disease, Classical Hodgkin Lymphoma, diffuse large B cell lymphoma, follicular lymphoma, Kaposi sarcoma, kidney cancer, laryngeal and hypopharyngeal cancer, leukemia (including acute myeloid leukemia), liver cancer (including hepatocellular carcinoma), lymphoma, melanoma (including unresectable or metastatic melanoma), prostate cancer, lung cancer (including non-small cell lung cancer and metastatic non-small cell lung cancer) malignant mesothelioma, merkel cell carcinoma, metastatic urothelial carcinoma, multiple myeloma, myeloma, myelodysplastic syndrome, nasal cavity and paranasal sinus cancer, nasopharyngeal cancer, neuroendocrine cancer, neuroblastoma, non-Hodgkin lymphoma, oral cavity and oropharyngeal cancer, osteosarcoma, ovarian cancer, pancreatic cancer, penile cancer, pituitary tumors, renal cancer (including renal cell carcinoma), retinoblastoma, hematological malignancy, rhabdomyosarcoma, salivary gland cancer, sarcoma, squamous cell skin cancer, small intestine cancer, stomach cancer, testicular cancer, thymus cancer, thyroid cancer, uterine cancer, and vaginal cancer.

92. The method of any one of claims 1-91, wherein the subject has received, is receiving, or will receive a cancer therapeutic.

93. The method of claim 92, wherein the cancer therapeutic is immunotherapy (e.g., adoptive cell therapy, cancer vaccine, immunomodulator, oncolytic virus therapy, or targeted antibody), a chemotherapy, a radiation therapy, a hormone therapy, a stem cell transplant, or a combination thereof.

94. The method of claim 92 or 93, wherein a sample is obtained around the time the subject is diagnosed a cancer or cancer recurrence or after the subject receives a cancer treatment, optionally after the subject completes one or more treatment cycles.

95. The method of any one of claims 92-94, wherein the sample is obtained after the subject completes one or more treatment cycles, optionally after the subject completes sufficient number of treatment cycles to stimulate a biomarker change in circulation.

96. The method of any one of claims 1-95, further comprising obtaining one or more samples from the subject and analyzing the biomarker.

97. The method of any one of claims 49 and 1-96, wherein the cancer therapeutic is selected from 5-fluorouracil, 6-mercaptopurine, 6-thioguanine, abemaciclib, abiraterone acetate, acalabrutinib, ado-trastuzumab emtansine, afatinib dimaleate, aldesleukin, alectinib, alemtuzumab, alpelisib, amifostine, aminolevulinic acid hydrochloride, anastrozole, apalutamide, arsenic trioxide, 1-asparaginase, atezolizumab, avelumab, axitinib, azacitidine, belinostat, bendamustine hydrochloride, bevacizumab, bexarotene, bicalutamide, binimetinib, bleomycin sulfate, blinatumomab, bortezomib, bosutinib, brentuximab vedotin, brigatinib, busulfan, cabazitaxel, cabozantinib-s-malate, calaspargase pegol-mknl, capecitabine, caplacizumab-yhdp, carboplatin, carfilzomib, carmustine, carmustine implant, cemiplimab-rwlc, ceritinib, cetuximab, chlorambucil, cisplatin, cladribine, clofarabine, cobimetinib, copanlisib hydrochloride, corticosteroids, crizotinib, cyclophosphamide, cytarabine, dabrafenib mesylate, dacarbazine, dacomitinib, dactinomycin, daratumumab, darolutamide, dasatinib, daunorubicin hydrochloride, daunorubicin hydrochloride and cytarabine liposome, decitabine, defibrotide sodium, degarelix, denileukin diftitox, denosumab, dexamethasone, dexamethasone, dexrazoxane hydrochloride, dinutuximab, docetaxel, doxorubicin hydrochloride, doxorubicin hydrochloride liposome, durvalumab, duvelisib, elotuzumab, eltrombopag olamine, emapalumab-lzsg, enasidenib mesylate, encorafenib, entrectinib, enzalutamide, epirubicin hydrochloride, erdafitinib, eribulin mesylate, erlotinib hydrochloride, etoposide, etoposide phosphate, everolimus, exemestane, fedratinib hydrochloride, fludarabine phosphate, flutamide, fostamatinib disodium, fulvestrant, gefitinib, gemcitabine hydrochloride, gemtuzumab ozogamicin, gilteritinib fumarate, glasdegib maleate, glucarpidase, goserelin acetate, hydroxyurea, ibritumomab tiuxetan, ibrutinib, idarubicin hydrochloride, idelalisib, ifosfamide, imatinib mesylate, imiquimod, inotuzumab ozogamicin, interferon alfa-2b, recombinant, iobenguane I 131, ipilimumab, irinotecan hydrochloride, irinotecan hydrochloride liposome, ivosidenib, ixabepilone, ixazomib citrate, lanreotide acetate, lapatinib ditosylate, larotrectinib sulfate, lenalidomide, lenvatinib mesylate, letrozole, leuprolide acetate, lomustine, lorlatinib, mechlorethamine hydrochloride, megestrol acetate, melphalan, methotrexate, methylnaltrexone bromide, methylprednisolone, midostaurin, mitomycin c, mitoxantrone hydrochloride, mogamulizumab-kpkc, moxetumomab pasudotox-tdfk, necitumumab, nelarabine, neratinib maleate, netupitant and palonosetron hydrochloride, nilotinib, nilutamide, niraparib tosylate monohydrate, nivolumab, obinutuzumab, ofatumumab, olaparib, omacetaxine mepesuccinate, osimertinib mesylate, oxaliplatin, paclitaxel, paclitaxel albumin-stabilized nanoparticle formulation, palbociclib, palifermin, panitumumab, panobinostat, pazopanib hydrochloride, pegaspargase, peginterferon alfa-2b, pembrolizumab, pemetrexed disodium, pertuzumab, polatuzumab vedotin-piiq, pomalidomide, ponatinib hydrochloride, pralatrexate, prednisone, procarbazine hydrochloride, propranolol hydrochloride, raloxifene hydrochloride, ramucirumab, ravulizumab-cwvz, recombinant interferon alfa-2b, regorafenib, ribociclib, rituximab, rituximab and hyaluronidase human, rolapitant hydrochloride, romidepsin, romiplostim, rucaparib camsylate, ruxolitinib phosphate, selinexor, siltuximab, sonidegib, sorafenib tosylate, sunitinib malate, tagraxofusp-erzs, talazoparib tosylate, tamoxifen citrate, temozolomide, temsirolimus, thalidomide, thiotepa, tocilizumab, topotecan hydrochloride, toremifene, trabectedin, trametinib, trastuzumab, trastuzumab and hyaluronidase-oysk, trifluridine and tipiracil hydrochloride, uridine triacetate, valrubicin, vandetanib, vemurafenib, venetoclax, vinblastine sulfate, vincristine sulfate, vincristine sulfate liposome, vinorelbine tartrate, vismodegib, vorinostat, zanubrutinib, and ziv-aflibercept.


SEQUENCE LISTING (2 sequences)

SEQ ID NO: 1 - protein, 107 aa, Homo sapiens
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 2 - protein, 14 aa, Artificial Sequence
  Description of Artificial Sequence Synthetic peptide
  MOD_RES (3)..(3): 3-(2-Naphthyl)-L-alanine
  MOD_RES (6)..(6): Citrulline
  MOD_RES (12)..(12): Citrulline
RRXCYXKKPYRXCR